(19) United States
(12) Patent Application Publication
(10) Pub. No.: US 2010/0041712 A1
(43) Pub. Date: Feb. 18, 2010

(54) SUBSTITUTED IMIDAZOLE COMBINATIONS

(75) Inventor: Nathan Anthony Logan Chubb, Richland Township, MI (US)

Correspondence Address: PHARMACIA & UPJOHN, 7000 Portage Road, KZO-300-104, KALAMAZOO, MI, 49001, US

(73) Assignee: PFIZER INC., New York, NY (US)

(21) Appl. No.: 12/491,507

(22) Filed: Jun. 25, 2009

Related U.S. Application Data
(60) Provisional application No. 61/089,815, filed on Aug. 18, 2008.

(51) Int. Cl. A61K 31/4402; A61K 31/4164; A61P 33/14

(52) U.S. Cl. 514/345; 514/396; 514/399

(57) ABSTRACT

This invention relates to a composition comprising a combination of a) an alpha substituted 2-benzyl substituted imidazole and b) a 1-N-arylpyrazole, and optionally c) an insect growth regulator, and their use as a parasiticide in mammals.

### CROSS-REFERENCE TO RELATED APPLICATIONS

This application claims benefit of priority from pending U.S. Provisional Application Ser. No. 61/089,815 filed Aug. 18, 2008.

### FIELD OF THE INVENTION

This invention relates to a veterinary composition which includes a combination of a substituted imidazole and substituted 1-N-arylpyrazole, and optionally an insect growth regulator and its use as a parasiticidal in mammals.

### BACKGROUND

There is a need for improved antiparasitic agents for use with mammals, preferably animals, and in particular there is a need for improved insecticides and acaricides. Furthermore there is a need for improved topical products with convenient administration and which contain one or more of such antiparasitic agents which can be used to effectively treat ectoparasites, such as insects and acarids, and particularly acarids such as mites and ticks. Such products would be particularly useful for the treatment of companion animals, such as cats, dogs and horses, and livestock, such as cattle. There is equally a need for agents to control parasitic infestations in animal hosts other than mammals, including insects such as bees, which are susceptible to parasites such as varroa mites.

The compounds currently available for insecticidal and acaricidal treatment of companion animals and livestock do not always demonstrate good activity, good speed of action, or a long duration of action. Most treatments contain hazardous chemicals that can have serious consequences, including lethality from accidental ingestion. Persons applying these agents are generally advised to limit their exposure. Pet collars and tags have been utilized to overcome some problems, but these are susceptible to chewing and ingestion. Thus, treatments currently achieve varying degrees of success which depend partly on toxicity, method of administration, and efficacy. Currently, some agents are actually becoming ineffective due to parasitic resistance.

Heterocyclic derivatives have been disclosed in the art as having insecticidal and acaricidal activity against agricultural pests, for example WO2003/092374.

Generic disclosures also exist in the art for heterocyclic derivatives which optionally encompass alpha substituted 2-benzyl imidazoles. For example, WO2005/007188 describes a generic structure that optionally encompasses alpha substituted 2-benzyl imidazoles for the inhibition of the hatching of an ectoparasite egg. Publication WO2004/103959 describes a generic structure that optionally encompasses alpha substituted 2-benzyl imidazoles for use as antibacterial agents. Publication WO2005/028425 describes a generic structure that optionally encompasses alpha substituted 2-benzyl imidazoles for use in the inhibition of chemotaxis of neutrophils induced by Interleukin-8. Publications WO01/00586 and: WO99/28300 both describe a generic structure that optionally encompasses alpha substituted 2-benzyl imidazoles and discloses their adrenergic activity. Still further, U.S. Pat. No. 6,103,733 describes a generic structure that optionally encompasses alpha substituted 2-benzyl imidazoles for increasing blood serum HDL cholesterol. However, none of these citations exemplify any alpha substituted 2-benzyl imidazoles, nor does the prior art indicate that such compounds would be useful against a spectrum of parasitic species relevant to companion animals and livestock or against the range of ectoparasite morphological lifecycle stages.

The present invention overcomes one or more of the various disadvantages of, and/or improves upon, the properties of existing compounds. In particular, it is the aim of the invention to develop a combination of compounds which includes alpha substituted 2-benzyl substituted imidazoles such as those described and claimed in Publication WO2007/083207, incorporated herein in its entirety by reference.

The efficacious combination of the present invention includes at least one alpha substituted 2-benzyl substituted imidazole, at least one substituted 1-N-arylpyrazole, e.g., 5-amino-1-[2,6-dichloro-4-(trifluoromethyl)phenyl]-4-(trifluoromethylsulfinyl)-1H-pyrazole-3-carbonitrile, commonly known as fipronil, and optionally an insect growth regulator, e.g., s-methoprene. Fipronil is a commercial product with the tradename “Frontline” (Merial, Lyons, France). Frontline Top Spot contains fipronil while Frontline Plus contains fipronil and s-methoprene. It is a further aim that such new combinations have improved activity when compared to the prior art combinations against parasites. It is another aim of the present invention to develop combinations which have a similar or decreased toxicity profile when compared to the prior art compounds. It is yet another aim to develop combinations which demonstrate selectivity for the octopaminergic receptor, a known invertebrate neurotransmitter, over the ubiquitous animal adrenergic receptor. Furthermore, it is an aim of the invention to reduce the exposure of both humans and animals to the treatment by developing compound combinations which can be dosed as a low volume spot-on or topical application. The combinations of the present invention have especially good ability to control against ticks and fleas and they are able to prevent ticks from attaching to, and feeding from, the host animal. It is yet another aim of the present invention to provide combinations which have good speed of action, improved duration of action, improved pharmacokinetic and safety profile, improved persistence, improved solubility and/or other improved physicochemical and formulation properties such as good spreading after topical application compared to those of the prior art.

### SUMMARY

The invention provides a combination of compounds described herein useful in a process for preventing, treating, repelling, and controlling tick, flea and mite infestation in mammals. When the combination includes s-methoprene, efficacy can be extended to control morphological stages of flea development. The combination with and without s-methoprene would also control and prevent flea allergy dermatitis. In addition, the invention contemplates a combination which aids in the control and/or prevention of tick borne diseases such as Lyme disease, canine anaplasmosis, canine ehrlichiosis, canine rickettsiosis and canine babesiosis.

Thus, according to the present invention, there is provided a combination which includes, an octopamine agonist, a) an alpha substituted 2-benzyl substituted imidazole of Formula (1):

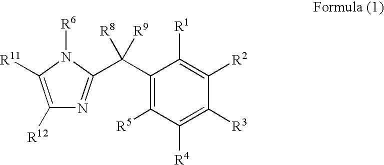

or a pharmaceutically or veterinarily acceptable salt thereof,
wherein:

R¹, R², R³, R⁴, R⁵ are independently selected from the group consisting of hydrogen, cyano, nitro, halo, hydroxy, C₁₋₄alkyl optionally substituted by one or more hydroxy groups, C₃₋₆ cycloalkyl optionally substituted by one or more C₁₋₄alkyl or halo groups, C₁₋₄alkoxy, C₁₋₄haloalkyl, C₁₋₄haloalkoxy, phenyl, amino, to NR^{x}R^{y}, and S(O)ₙP¹⁰;

R⁶ is selected from the group consisting of hydrogen, —C₀₋₂alkyleneR⁷, —C₁₋₂alkyleneOR⁷, —C₀₋₂alkyleneC(O)R⁷, —C₁₋₂alkyleneOC(O)R⁷, —C₁₋₂alkyleneOC(O)OR⁷, —C₀₋₂alkyleneC(O)OR⁷, —C₁₋₂alkyleneN(H)C(O)R⁷, C₁₋₂alkyleneN(R⁷)C(O)R⁷, —C₀₋₂alkyleneC(O)NHR⁷, —C₀₋₂alkyleneC(O)NR¹⁵R¹⁶, —C₁₋₂alkyleneNHC(O)NR¹⁵R¹⁶, —C₁₋₂alkyleneNR⁷C(O)NR¹⁵R¹⁶, —C₁₋₂alkyleneOC(O)NHR⁷, —C₁₋₂alkyleneOC(O)NR¹⁵R¹⁶, —C₀₋₂alkyleneCH═N(R⁷), —C₁₋₂alkyleneP(═O)(NR¹⁵R¹⁶)(NR¹⁵R¹⁶), —C₀₋₂alkyleneSi(R⁷)₃, and —C₀₋₂alkyleneS(O)ₙR¹⁰;

where the C₀₋₂alkylene or C₁₋₂alkylene of R⁶ may, where chemically possible, optionally be substituted by one or more substituents selected from the group consisting of C₁₋₆ alkyl, C₃₋₆ cycloalkyl, C₁₋₄ alkylene(C₃₋₆ cycloalkyl), C₀₋₆ alkylenephenyl, which C₀₋₂alkylene or C₁₋₂alkylene substituent may in turn be optionally further substituted, where chemically possible, by one or more substituents selected from the group consisting of hydrogen, cyano, nitro, halo, formyl, oxo, hydroxy, C(O)OH, C₁₋₄ alkyl, C₁₋₄ alkyleneC₃₋₅ cycloalkyl, C₁₋₄ alkoxy, C₁₋₄ alkyleneC₁₋₄ alkoxy, —C(O)OC₁₋₄ alkyl, C₁₋₄ haloalkyl, C₁₋₄ haloalkoxy, amino, C₁₋₄ alkylamino, C₁₋₄ dialkylamino, and S(O)ₙR¹⁰;

where each R⁷, R¹⁵ and R¹⁶, where chemically possible, is independently selected from the group consisting of hydrogen, C₁₋₈ alkyl, C₂₋₆ alkenyl, C₂₋₆ alkynyl, C₃₋₈ cycloalkyl, C₁₋₄ alkylene(C₃₋₆ cycloalkyl), C₁₋₄ alkyleneC₁₋₄ alkoxy, C₁₋₆ haloalkyl, C₀₋₆ alkylenephenyl, C₀₋₆ alkylenenaphthyl, C₀₋₆ alkylene(tetrahydro-naphthyl), and C₀₋₂ alkylene(Het), where Het is selected from oxetanyl, tetrahydropyranyl, piperidinyl, morpholinyl, furyl, pyridyl, benzofuranyl, benzothiazolyl, indolyl, 2,3-benzodioxolyl, 2,3-dihydro-1,4-benzodioxinyl, indolyl and 1,5-naphthyridinyl;

or R¹⁵ and R¹⁶ together with the nitrogen to which they are attached may form a three- to seven-membered saturated or unsaturated heterocyclic ring optionally containing one or more further N, O or S atoms or SO₂ groups;

where each of the above R⁷, R¹⁵ or R¹⁶ groups may independently include one or more optional substituents where chemically possible selected from hydrogen, cyano, nitro, halo, formyl, oxo, hydroxy, C(O)OH, C₁₋₄ alkyl, C₂₋₄ alkenyl, C₂₋₄ alkynyl, C₃₋₆ cycloalkyl, C₁₋₄ alkyleneC₃₋₆ cycloalkyl, C₁₋₄ alkoxy, C₁₋₄ alkyleneC₁₋₄ alkoxy, C₁₋₄ alkoxyC₁₋₄ alkoxy, C₁₋₄ alkanoyl, —C(O)OC₁₋₄ alkyl, C₁₋₄ haloalkyl, C₃₋₆ halocycloalkyl, C₁₋₄ haloalkoxy, C₁₋₄ haloalkanoyl, —C(O)OC₁₋₄ haloalkyl, phenyl, 4-halophenyl, 4-alkoxyphenyl, 2-cyanophenyl, phenoxy, 4-halophenoxy, benzyloxy, 4-halobenzyloxy, benzoyl, pyrazolyl, triazolyl, 2-halo-4-pyrimidinyl, 2-phenylethyl, amino, C₁₋₄ alkylamino, C₁₋₄ dialkylamino, C(O)N(C₁₋₄ alkyl)₂, N(C₁₋₄ alkylene)C(O)(C₁₋₄ alkyl) and S(O)ₙR¹⁰;

R⁸ and R⁹ are independently selected from the group consisting of hydrogen, C₁₋₄ alkyl, C₁₋₄ alkoxy, C₁₋₄ haloalkyl, C₁₋₄ haloalkoxy and C₀₋₄ alkylenephenyl but with the proviso that R⁸ and R⁹ are not both hydrogen;

where each of R⁸ and R⁹ may independently include one or more optional substituents where chemically possible selected from hydrogen, cyano, halo, hydroxy, C₁₋₄ alkyl, C₃₋₆ cycloalkyl, C₁₋₄ alkoxy, —C(O)C₁₋₄ alkyl, C₁₋₄ haloalkyl, C₁₋₄haloalkoxy, and S(O)ₙR¹⁰;

or R⁸ and R⁹ together with the carbon to which they are attached may form a three- to six-membered carbocyclic, saturated ring, which ring is optionally substituted with one or more substituents selected from the group consisting of halo, C₁₋₂ alkyl, C₁₋₂ alkoxy, C₁₋₂ haloalkyl, C₁₋₂ haloalkoxy;

R¹¹ and R¹² are independently selected from the group consisting of hydrogen, halo, cyano, C₁₋₄ alkyl, C₁₋₄ alkoxy, C₁₋₄ haloalkyl, and C₁₋₄ haloalkoxy; where R^{x} and R^{y} are independently selected from hydrogen, C₁₋₄ alkyl, C₁₋₄ haloalkyl, and S(O)ₙR¹⁰;

each n is independently 0, 1 or 2; and

each R¹⁰ is independently hydrogen, hydroxy, C₁₋₄ alkyl, C₁₋₄ haloalkyl, 4-halophenyl, amino, C₁₋₆ alkyl amino and di C₁₋₆ alkylamino; and

b) a substituted 1-N-arylpyrazole of Formula (X)

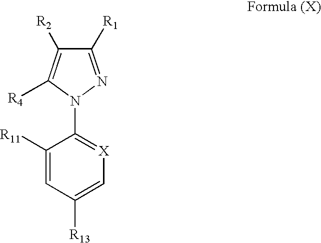

or a pharmaceutically or veterinarily acceptable salt thereof,
wherein

R₁ is CN or methyl or a halogen atom;

R₂ is S(O)ₙR₃ or 4,5-dicyanoimidazol-2-yl or haloalkyl;

R₃ is alkyl or haloalkyl;

R₄ represents a hydrogen or halogen atom; or a member of a group consisting of NR₅R₆, S(O)ₘR₇, C(O)R₇, C(O)O—R₇, alkyl, haloalkyl, OR₈ and —N═C(R₉) (R₁₀);

R₅ and R₆ independently represent a hydrogen atom or an alkyl, haloalkyl, C(O)alkyl, alkoxycarbonyl or S(O)ᵣF₃ radical; or R₅ and R₆ may together form a divalent alkylene radical which may be interrupted by one or two divalent hetero atoms such as oxygen or sulphur;

P₇ represents an alkyl or haloalkyl radical;

R₈ represents an alkyl or haloalkyl radical or a hydrogen atom;

R₉ represents an alkyl radical or a hydrogen atom;

R₁₀ represents a phenyl or heteroaryl group optionally substituted with one or more halogen atoms or a member of the group consisting of OH, —O-alkyl, S-alkyl, cyano and alkyl;

R₁₁ and R₁₂ represent, independently of each other, a hydrogen or halogen atom, or possibly CN or NO₂;

R₁₃ represents a halogen atom or a haloalkyl, haloalkoxy, S(O)_{q}CF₃ or SF₅ group;

m, n, q and r represent, independently of each other, an integer equal to 0, 1 or 2; and

X represents a trivalent nitrogen atom or a radical C—R₁₂, the other three valency positions of the carbon atom forming part of the aromatic ring,

with the proviso that when R₁ is methyl, either R₃ is haloalkyl, R₄ is NH₂, R₁₁ is Cl, R₁₃ is CF₃ and X is N; or R₂ is 4,5-dicyanoimidazol-2-yl, R₄ is Cl, R₁₁ is Cl, R₁₃ is CF₃ and X is ═C—Cl;

and optionally c) an insect growth regulator which mimics juvenile hormones which is hydroprene, s-methoprene, or pyriproxyfen.

In the definition of R¹, R², R³, R⁴ and R⁵ of Formula (1) compounds, “C₁₋₄ alkyl optionally substituted by one or more hydroxy groups” means an alkyl group with between one and four carbon atoms, which may be unsubstituted or may be substituted at any available position with a hydroxy group. For reasons of chemical stability, it is preferred that no carbon atom should be substituted with more than one hydroxy group. Accordingly, alkyl groups with up to four hydroxy substituents are foreseen. Preferred are alkyl groups with no more than two hydroxy substituents. Examples include hydroxymethyl, 1-hydroxyethyl, 2-hydroxyethyl, 1,2-dihydroxyethyl and 2,3-dihydroxypropyl.

In the definition of R¹, R², R³, R⁴ and R⁵ of Formula (1) compounds, “C₃₋₆ cycloalkyl optionally substituted by one or more C₁₋₄ alkyl or halo groups” means a cycloalkyl group with between three and six carbon atoms in the ring, which may be unsubstituted or may be substituted at any available position with an alkyl group of between one and four carbon atoms or a halogen atom. In the case of alkyl substituents, it is preferred that not more than four such substituents be present, and more preferred that not more than two such substituents be present. Examples include 1-methylcyclopropyl, 2,5-dimethylcyclopentyl and 4-tert-butylcyclohexyl. In the case of halo substituents, any degree of substitution up to complete substitution is foreseen. In the case of cyclohexyl therefore, up to eleven halo substituents may be present. While each halo group may be independently selected, it may be preferred to have all halo substituents the same. Preferably the halo is chloro or fluoro. Geminal disubstitution at any methylene position may be preferred over monosubstitution. Examples include 2,2dichlorocyclopropyl and perfluorocyclohexyl. Substitution with both alkyl and halo groups is also foreseen. An example is 2,2-difluoro-1-methylcyclobutyl.

In particular, the alpha substituted 2-benzyl substituted imidazole is a Formula (1) compound:

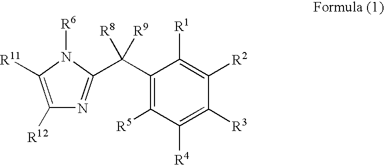

or a pharmaceutically or veterinarily acceptable salt thereof.
wherein:

R¹, R², R³, R⁴, R⁵ are independently selected from the group consisting of hydrogen, cyano, nitro, halo, hydroxy, C₁₋₄ alkyl, C₃₋₆ as cycloalkyl, C₁₋₄ alkoxy, C₁₋₄ haloalkyl, C₁₋₄ haloalkoxy, amino, NR^{x}R^{y}, and S(O)ₙR¹⁰;

R⁶ is selected from the group consisting of hydrogen, —C₀₋₂alkyleneR⁷, C₁₋₂alkyleneOR⁷, —C₀₋₂alkyleneC(O)R⁷, C₁₋₂alkyleneOC(O)R⁷, C₁₋₂alkyleneOC(O)OR⁷, —C₀₋₂alkyleneC(O)OR⁷, C₁₋₂alkyleneN(H)C(O)R⁷, —C₁₋₂alkyleneN(R⁷)C(O)R⁷, —C₀₋₂alkyleneC(O)NHR⁷, —C₀₋₂alkyleneC(O)NR¹⁵R¹⁶, —C₁₋₂alkyleneNHC(O)NR¹⁵R¹⁶, —C₁₋₂alkyleneNR⁷C(O)NR¹⁵R¹⁶, —C₁₋₂alkyleneOC(O)NHR⁷, —C₁₋₂alkyleneOC(O)NR¹⁵R¹⁶, —C₀₋₂alkyleneCH═N(R⁷), —C₁₋₂alkyleneP(═O)(NR¹⁵R¹⁶)(NR¹⁵R⁶), —C₀₋₂alkyleneSi(R⁷)₃, and —C₀₋₂alkyleneS(O)ₙR¹⁰;

where the C₀₋₂alkylene or C₁₋₂alkylene of R⁶ may, where chemically possible, optionally be substituted by one or more substituents selected from the group consisting of C₁₋₆ alkyl, C₃₋₆ cycloalkyl, C₁₋₄ alkylene(C₃₋₆ cycloalkyl), C₀₋₆ alkylenephenyl, which C₀₋₂alkylene or C₁₋₂alkylene substituent may in turn be optionally further substituted, where chemically possible, by one or more substituents selected from the group consisting of hydrogen, cyano, nitro, halo, formyl, oxo, hydroxy, C(O)OH, C₁₋₄ alkyl, C₁₋₄ alkyleneC₃₋₆ cycloalkyl, C₁₋₄ alkoxy, C₁₋₄ alkyleneC₁₋₄ alkoxy, —C(O)OC₁₋₄ alkyl, C₁₋₄ haloalkyl, C₁₋₄ haloalkoxy, amino, C₁₋₄ alkylamino, C₁₋₄ dialkylamino, and S(O)ₙPR¹⁰;

where each R⁷, R¹⁵ and R¹⁶, where chemically possible, is independently selected from the group consisting of hydrogen, C₁₋₈ alkyl, C₂₋₆ alkenyl, C₂₋₆ alkynyl, C₃₋₈ cycloalkyl, C₁₋₄ alkylene(C₃₋₆ cycloalkyl), C₁₋₄ alkyleneC₁₋₄ alkoxy, C₁₋₆ haloalkyl, C₀₋₆ alkylenephenyl;

or R¹⁵ and R¹⁶ together with the nitrogen to which they are attached may form a three- to seven-membered saturated or unsaturated heterocyclic ring optionally containing one or more further N, O or S atoms;

where each of the above R⁷, R¹⁵ or R¹⁶ groups may independently include one or more optional substituents where chemically possible selected from hydrogen, cyano, nitro, halo, formyl, oxo, hydroxy, C(O)OH, C₁₋₄ alkyl, C₂₋₄ alkenyl, C₂₋₄ alkynyl, C₃₋₆ cycloalkyl, C₁₋₄ alkyleneC₃₋₆ cycloalkyl, C₁₋₄ alkoxy, C₁₋₄ alkyleneC₁₋₄ alkoxy, C₁₋₄ alkanoyl, —C(O)OC₁₋₄ alkyl, C₁₋₄ haloalkyl, C₃₋₆ halocycloalkyl, C₁₋₄ haloalkoxy, C₁₋₄ haloalkanoyl, —C(O)OC₁₋₄ haloalkyl, phenyl, 4-halophenyl, 4-alkoxyphenyl, amino, C₁₋₄ alkylamino, C₁₋₄ dialkylamino, C(O)N(C₁₋₄ alkyl)₂, N(C₁₋₄ alkylene)C(O)(C₁₋₄ alkyl) and S(O)ₙR¹⁰;

R⁸ and R⁹ are independently selected from the group consisting of hydrogen, C₁₋₄ alkyl, C₁₋₄ alkoxy, C₁₋₄ haloalkyl, C₁₋₄ haloalkoxy and C₀₋₄ alkylenephenyl but with the proviso that R⁸ and ⁹ are not both hydrogen;

where each of R⁸ and R⁹ may independently include one or more optional substituents where chemically possible selected from hydrogen, cyano, halo, hydroxy, C₁₋₄ alkyl, C₃₋₆ cycloalkyl, C₁₋₄ alkoxy, —C(O)OC₁₋₄ alkyl, C₁₋₄ haloalkyl, C₁₋₄ haloalkoxy, and S(O)ₙR¹⁰;

or R⁸ and R⁹ together with the carbon to which they are attached may form a three- to six-membered carbocyclic, saturated ring, which ring is optionally substituted with one or more substituents selected from the group consisting of halo, C₁₋₂ alkyl, C₁₋₂ alkoxy, C₁₋₂ haloalkyl, C₁₋₂ haloalkoxy;

R¹¹ and R¹² are independently selected from the group consisting of to hydrogen, halo, cyano, C₁₋₄ alkyl, C₁₋₄ alkoxy, C₁₋₄ haloalkyl, and C₁₋₄ haloalkoxy;

where R^{x} and R^{y} are independently selected from hydrogen, C₁₋₄ alkyl, C₁₋₄ haloalkyl, and S(O)ₙR¹⁰;
each n is independently 0, 1 or 2;
and each R¹⁰ is independently hydrogen, hydroxy, C₁₋₄ alkyl, C₁₋₄ haloalkyl, amino, C₁₋₆ alkyl amino and di C₁₋₆ alkyl amino;

Preferably, each of R¹, R², R³, R⁴, R⁵ are independently selected from hydrogen, halo (e.g., chloro or fluoro), C₁₋₄ alkyl (e.g., methyl or ethyl), C₃₋₄ cycloalkyl (e.g., cyclopropyl), C₁₋₄ alkoxy (e.g., methoxy or ethoxy), C₁₋₄ haloalkyl (e.g., trifluoromethyl, trifluoroethy), C₁₋₄ haloalkoxy (e.g., trifluoromethoxy or trifluoroethoxy), and S(O)ₙR¹⁰ where n is 0 and R¹⁰ is preferably selected from C₁₋₄ alkyl such as methyl or ethyl or C₁₋₄ haloalkyl such as trifluoromethyl or trifluoroethyl to form for example trifluoromethylthio or trifluoroethylthio. More preferably each of R¹, R², R³, R⁴, R⁵ are independently selected from hydrogen, halo (e.g., chloro), C₁₋₄ alkyl (e.g., methyl or ethyl), C₁₋₄ alkoxy (e.g., methoxy or ethoxy), and C₁₋₄ haloalkyl (e.g., trifluoromethyl, trifluoroethyl). Most preferably each of R¹, R², R³, R⁴, R⁵ are independently selected from hydrogen, and C₁₋₄ alkyl (e.g., methyl or ethyl).

Most preferably two of R¹, R², R³, R⁴, and R⁵ are independently selected from C₁₋₄ alkyl (e.g., methyl or ethyl), preferably methyl, and three of R¹, R², R³, R⁴, and R⁵ are H. Even more preferably R¹ and R² are selected from C₁₋₄ alkyl (e.g., methyl or ethyl), preferably methyl, and R³, R⁴ and R⁵ are hydrogen.

Preferably R⁶ is selected from the group consisting of hydrogen, —C₀₋₂alkyleneR⁷, —C₁₋₂alkyleneOR⁷, —C₁₋₂alkyleneOC(O)R⁷, —C₁₋₂alkyleneOC(O)OR^{7,} —C₀₋₂alkyleneC(O)OR⁷, —C₁₋₂alkyleneOC(O)NH⁷, —C₁₋₂alkyleneOC(O)NR¹⁵R¹⁶, and —C₀₋₂alkyleneS(O)ₙR¹⁰. More preferably R⁶ is selected from the group consisting of hydrogen, —C₀₋₂alkyleneR⁷, —C₁₋₂alkyleneOR⁷, —C₁₋₂alkyleneOC(O)R⁷, —C₁₋₂alkyleneOC(O)OR⁷, and —C₀₋₂alkyleneC(O)OR⁷. Even more preferably R⁶ is selected from the group consisting of hydrogen, —C₀₋₂alkyleneR⁷, —C₁₋₂alkyleneOC(O)R⁷ and —C₀₋₂alkyleneC(O)OR⁷. Most preferably R⁶ is hydrogen.

Preferably R⁷, R¹⁵ and R¹⁶ are, where chemically possible, independently selected from the group consisting of hydrogen, C₁₋₈ alkyl for example methyl, ethyl, n-propyl, isopropyl, butyl, isobutyl, tert-butyl, n-pentyl, n-hexyl; C₃₋₈ cycloalkyl for example cyclopropyl, cyclobutyl, cyclopentyl, cyclohexyl; C₁₋₄ alkylene(C₃₋₆ cycloalkyl) for example cyclopropylmethyl, cyclopropylethyl, cyclobutylmethyl, cyclobutylethyl, cyclopentylmethyl, cyclopentylethyl, cyclohexylmethyl, cyclohexylethyl; C₁₋₆ haloalkyl for example fluoromethyl, trifluoromethyl, chloromethyl, fluoroethyl, chloroethyl, trifluoroethyl and trifluoropropyl; and C₀₋₆ alkylphenyl for example phenyl, phenylmethyl and phenylethyl. More preferably R⁷, R¹⁵ and R¹⁶ are, where chemically possible, independently selected from the group consisting of hydrogen; C₁₋₆ alkyl for example methyl, ethyl, n-propyl, isopropyl, butyl, tert-butyl, n-pentyl, n-hexyl; C₁₋₄ alkylene(C₃₋₆ cycloalkyl) for example cyclopropylmethyl, cyclopropylethyl, cyclobutylmethyl, cyclobutylethyl, cyclopentylmethyl, cyclopentylethyl, cyclohexylmethyl, cyclohexylethyl. Even more preferably R⁷, R¹⁵ and R¹⁶ are, where chemically possible, independently selected from the group consisting of hydrogen and C₁₋₄ alkyl for example methyl, ethyl, propyl, isopropyl, n-butyl and tert-butyl Preferably, each R⁸ and R⁹ are independently selected from the group consisting of hydrogen; C₁₋₄ alkyl (e.g., methyl or ethyl), preferably methyl; C₁₋₄ haloalkyl for example trifluoromethyl, trichloromethyl, trichloroethyl or trifluoroethyl, preferably trifluoromethyl; C₁₋₄ alkoxy for example methoxy or ethoxy, preferably methoxy; and C₀₋₄ alkylenephenyl for example phenyl, phenylmethyl or phenylethyl, but with the proviso that R⁸ and R⁹ are not both hydrogen. More preferably each R⁸ and R⁹ are independently selected from the group consisting of hydrogen and C₁₋₄ alkyl (e.g., methyl or ethyl), preferably methyl but again with the proviso that R⁸ and R⁹ are not both hydrogen. Most preferably, R⁸ is methyl and R⁹ is hydrogen.

Preferably each of R¹¹ and R¹² are independently selected from the group consisting of hydrogen, C₁₋₂ alkyl (e.g., methyl or ethyl), preferably methyl, and C₁₋₂ alkoxy for example methoxy or ethoxy, preferably methoxy. More preferably at least one of R¹¹ and R¹² is hydrogen. Most preferably both of R¹¹ and R¹² are hydrogen.

Preferred Formula I compounds include those of Formula (IA) and Formula (IB) which possess the stereochemistry shown below.

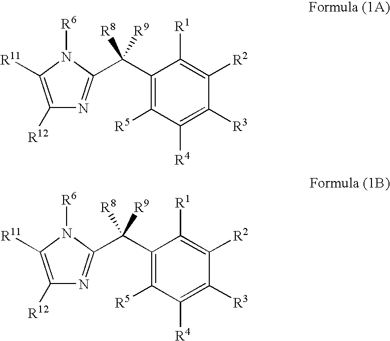

The variables R¹⁻⁶, R⁸⁻⁹, and R¹¹⁻¹² for formula's (1A) and (1B) are as described above for Formula (1).

Even more preferred Formula (1A) and (1B) compounds are compounds (1A1) and (1B1) shown below

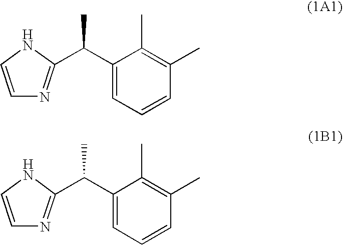

which are 2-[(1S)-1-(2,3-dimethylphenyl)ethyl]-1H-imidazole (1A1) and 2-[(1R)-1-(2,3-dimethylphenyl)ethyl]-1H-imidazole (1B1), or a pharmaceutically or veterinarily acceptable salt thereof. The most preferred is 2-[(1S)-1-(2,3-dimethylphenyl)ethyl]-1H-imidazole (1A1).

### Suitable Compounds of Formula (1)

Further suitable compounds include those where at least one of R¹, R², R³, R⁴, and R⁵ is independently C₁₋₄ haloalkyl, e.g., trifluoromethyl, trifluoroethyl, preferably trifluoromethyl; with the others of R¹, R², R³, R⁴, and R⁵ being H. Preferably R² is C₁₋₄ haloalkyl, e.g., trifluoromethyl or trifluoroethyl, preferably trifluoroethyl, with the others of R¹, R³, R⁴, and R⁵ being H.

Other suitable compounds include those where at least one of R¹, R², R³, R⁴, and R⁵ is independently selected from C₁₋₄ alkoxy, e.g., methoxy or ethoxy, preferably methoxy, with the others of R¹, R², R³, R⁴, and R⁵ being H. Preferably R² and R³ are selected from C₁₋₄ alkoxy, e.g., methoxy or ethoxy, preferably methoxy, and R¹, R⁴ and R⁵ are H.

Other suitable compounds include those where at least one of R¹, R², R³, R⁴, and R⁵ is independently halo, e.g., chloro or fluoro, with the others of R¹, R², R³, R⁴, and R⁵ being H.

Other suitable compounds include those where at least one of R¹, R², R³, R⁴, and R⁵ is independently halo, e.g., chloro or fluoro, and another one of R¹, R², R³, R⁴, and R⁵ is independently C₁₋₄ alkyl, e.g., methyl or ethyl, with the others of R¹, R², R³, R⁴, and R⁵ being H.

Suitable compounds also include those where, when the R⁶ group comprises a one carbon alkylene moiety, that said alkylene moiety is optionally substituted with one or two substituents. Further suitable compounds also include those where, when the R⁶ group comprises a two carbon alkylene moiety, that said alkylene moiety is optionally substituted with one, two, three or four substituents which may be independently orientated on either the alpha or beta carbon positions with respect to the imidazole nitrogen to which the R⁶ substitutent is bound.

Suitably when the C₀₋₂alkylene or C₁₋₂alkylene of R⁶ is substituted with one or more substitutents it is preferred that such substituents are independently selected from the group consisting of hydrogen; C₁₋₄ alkyl, e.g., methyl or ethyl; C₃₋₆ cycloalkyl, e g., cyclopropyl; C₁₋₄ alkyleneC₃₋₆ cycloalkyl, e.g., cyclopropylmethyl or cyclopropylethyl; C₁₋₄ alkoxy, e.g., methoxy or ethoxy; C₁₋₄ alkyleneC₁₋₄alkoxy, e.g., methoxymethyl, methoxyethyl, ethoxymethyl or ethoxyethyl; C₁₋₄ haloalkyl, e.g., fluoromethyl, trifluromethyl, fluoroethyl or 1,1,1-trifluoroethyl; phenyl, benzyl and 4-trifluoromethylbenzyl. More preferably such substituents are independently chosen from hydrogen, methyl, ethyl, cyclopropyl, cyclopropyl methyl, cyclopropylethyl, fluoromethyl, trifluromethyl, fluoroethyl, 1,1,1-trifluoroethyl, and phenyl.

Suitable compounds also include those where R⁶ is selected from the group consisting of —C₀₋₂alkyleneR⁷, preferably where R⁶ is CH₂R⁷, and where R⁷ is selected from the group consisting of C₁₋₈ alkyl, e.g., methyl, ethyl, n-propyl, isopropyl, butyl, tert-butyl; C₃₋₈ cycloalkyl, e.g., cyclopropyl, cyclobutyl, and cyclopentyl; C₁₋₆ haloalkyl, e.g., trifluoromethyl and trifluoroethyl; and C₀₋₆ alkylenephenyl, e.g., phenyl which is optionally substituted to form, e.g., 4-methoxyphenyl or 4-trifluoromethylphenyl. Further suitable compounds also include those where R⁶ is selected from the group consisting of —C₀₋₂alkyleneR⁷, preferably where R⁶ does not comprise an additional alkylene moiety (i.e., is C₀alkyleneR⁷), and where R⁷ is selected from the group consisting of C₁₋₈ alkyl, e.g., methyl, ethyl, n-propyl, isopropyl, butyl, tert-butyl; C₃₋₈ cycloalkyl, e.g., cyclopropyl, cyclobutyl, and cyclopentyl; C₁₋₆ haloalkyl, e.g., trifluoromethyl and trifluoroethyl; and C₀₋₆ alkylenephenyl, e.g., phenyl which is optionally substituted to form, e.g., 4-methoxyphenyl and 4-trifluoromethylphenyl.

A further group of suitable compounds include those where R⁶ is selected from the group consisting of —C₁₋₂alkyleneOR⁷, preferably where R⁶ is CH₂OR⁷, and where R⁷ is selected from the group consisting of C₁₋₈ alkyl. Examples of such so substituted R⁶ groups include methoxymethyl, ethoxymethyl, methoxyethyl, ethoxyethyl, propoxymethyl, propoxyethyl, isopropoxyethyl, butoxymethyl, sec-butoxyoxymethyl, isobutoxymethyl, tert-butoxymethyl, butoxyethyl, sec-butoxyoxyethyl, isobutoxyethyl, tert-butoxyethyl, pentloxymethyl, pentyloxyethyl, hexyloxymethyl, and hexyloxyethyl.

A still further group of suitable compounds include those where R⁶ is selected from the group consisting of —C₁₋₂alkyleneOC(O)R⁷, preferably where R⁶ is CH₂CO(O)R⁷, and where R⁷ is C₁₋₈ alkyl which R⁷ in turn may be optionally further substituted. Examples of such so substituted R⁶ groups include acetyloxymethyl, acetyloxyethyl, propionyloxymethyl, propionyloxyethyl, butyryloxymethyl, butyryloxyethyl, isobutyryloxymethyl, isobutyryloxyethyl, pentanoyloxymethyl, pentanoyloxyethyl, 2-methylbutyryloxymethyl, 2-methylbutyryloxyethyl, 3-methylbutyryloxymethyl, 3-methylbutyrylcarbonyloxy)-ethyl, 2,2-dimrethylpropionyloxymethyl, 2,2-dimethylpropionyloxyethyl hexanoyloxymethyl, hexanoyloxyethyl, heptanoyloxymethyl, heptanoyloxyethyl Further suitable examples of compounds where R⁶ is selected from the group consisting of —C₁₋₂alkyleneOC(O)R⁷, preferably where R⁶ is CH₂OC(O)R⁷, also include those where R⁷ is C₁₋₄ alkylene(C₃₋₆ cycloalkyl), e.g., cyclopropylmethyl, cyclopropylethyl, cyclobutylmethyl, cyclobutylethyl, cyclopentylmethyl, cyclopentylethyl, cyclopentylpropyl, cyclohexylmethyl, and cyclcohexylethyl. Examples of such so substituted R⁶ groups include cyclopropylacetyloxymethyl, cyclopropylpropionyloxymethyl, cyclobutylacetyloxymethyl, cyclobutylpropionyloxymethyl, cyclopentylacetyloxymethyl, cyclopentylpropionyloxymethyl, cyclopentylbutyryloxymethyl, cyclohexylacetyloxymethyl, and cyclcohexylpropionyloxymethyl, cyclopropylacetyloxyethyl, cyclopropylpropionyloxyethyl, cyclobutylacetyloxyethyl, cyclobutylpropionyloxyethyl, cyclopentylacetyloxyethyl, cyclopentylpropionyloxyethyl, cyclopentylbutyryloxyethyl, cyclohexylacetyloxyethyl, and cyclcohexylpropionyloxyethyl. Preferably R⁶ is 3-cyclopentylpropionyloxymethyl. It is preferred that in such compounds R⁷ is preferably C₁₋₈ alkyl, more preferably ethyl or tert-butyl, and most preferably tert-butyl.

A yet further group of suitable compounds include those where R⁶ is selected from the group consisting of —C₁₋₂alkyleneOC(O)OR⁷, preferably where R⁶ is CH₂OC(O)OR⁷, and where R⁷ is C₁₋₈ alkyl which may in turn be optionally further substituted. Examples of such so substituted R⁶ groups include methoxycarbonyloxymethyl, methoxycarbonyloxyethyl, ethoxycarbonyloxymethyl, ethoxycarbonyloxyethyl, propoxycarbonyloxymethyl, propoxycarbonyloxyethyl, isopropoxycarbonyloxymethyl, isopropoxycarbonyloxyethyl, butoxycarbonyloxymethyl, butoxycarbonyloxyethyl, isobutoxycarbonyloxymethyl, isobutoxycarbonyloxyethyl, pentyloxycarbonyloxymethyl, pentyloxycarbonyloxyethyl, 2-methyl butoxycarbonyloxymethyl, 2-methylbutoxycarbonyloxyethyl, 3-methylbutoxycarbonyloxymethyl, 3-methylbutoxycarbonyloxyethyl, 2,2-dimethylpropoxycarbonyloxymethyl, 2,2-dimethylpropoxycarbonyloxyethyl, hexyloxycarbonyloxymethyl, hexyloxycarbonyloxyethyl. Further suitable examples of compounds where R⁶ is selected from the group consisting of —C₁₋₂alkyleneOC(O)OR⁷, preferably where R⁶ is CH₂OC(O)OR⁷, also include those where R⁷ is selected from the group consisting of C₃₋₆ cycloalkyl for example cyclopropyl, cyclobutyl, cyclopentyl or cyclohexyl; C₁₋₄ alkylene(C₃₋₆ cycloalkyl), e.g., cyclopropylmethyl, cyclopropylethyl, cyclobutylmethyl, cyclobutylethyl, cyclopentylmethyl, cyclopentylethyl, cyclohexylmethyl, cyclohexylethyl; C₁₋₆ haloalkyl for example trifluoromethyl, and 2,2,2-trifluoroethyl; and C₀₋₆ alkylphenyl, e.g., phenyl which is optionally further substituted to form for example 4-methoxyphenyl or 4-trifluoromethylphenyl4-methoxybenzyl. Examples of such so substituted R₆ groups include cyclopropyloxycarbonyloxymethyl, cyclobutyloxycarbonytoxymethyl, cyclopentyloxycarbonyloxymethyl or cyclohexyloxycarbonytoxymethyl cyclopropyloxycarbonyloxyethyl, cyclobutyloxycarbonyloxyethyl, cyclopentyloxycarbonyloxyethyl or cyclohexyloxycarbonyloxyethyl; C₁₋₄ alkylene(C₃₋₆ cycloalkyl) for example cyclopropylmethyloxycarbonyloxymethyl, cyclopropylethyloxycarbonyloxymethyl, cyclobutylmethyloxycarbonyloxymethyl, cyclobutylethyloxycarbonyloxymethyl, cyclopentylmethyloxycarbonyloxymethyl, cyclopentylethyloxycarbonyloxymethyl, cyclohexylmethyloxy, carbonyloxymethyl, cyclohexylethyloxycarbonyloxymethyl, cyclopropylmethyloxycarbonyloxyethyl, cyclopropylethyloxycarbonyloxyethyl, cyclobutylmethyloxycarbonyloxyethyl, cyclobutylethyloxycarbonyloxyethyl, cyclopentylmethyloxycarbonyloxyethyl, cyclopentylethyloxycarbonyioxyethyl, cyclohexylmethyloxycarbonyloxyethyl, cyclohexylethyloxycarbonyloxyethyl; C₁₋₆ haloalkyl for example trifluoromethyloxycarbonyloxymethyl, and 2,2,2-trifluoroethyloxycarbonyloxymethyl, trifluoromethyloxycarbonyloxyethyl, and 2,2,2-trifluoroethytoxycarbonyloxyethyl; and C₀₋₆ alkylphenyl for example phenyloxycarbonyloxymethyl which is optionally further substituted to form for example 4-methoxyphenyloxycarbonyloxymethyl, 4-trifluoromethylphenyloxycarbonyloxymethyl, 4-methoxybenzyloxycarbonyloxymethyl.

A still yet further group of suitable compounds include those where R⁶ is selected from the group consisting of —C₀₋₂alkylene C(O)OR⁷, preferably where R⁶ is C(O)OR⁷, and where R⁷ is C₁₋₈ alkyl which may in turn be optionally further substituted Examples of such so substituted R⁶ groups include methoxycarbonyl₅ methoxycarbonylmethyl, methoxycarbonylethyl, ethoxycarbonyl, ethoxycarbonylmethyl, ethoxycarbonylethyl, propoxycarbonyl, propoxycarbonylmethyl, propoxycarbonylethyl, isopropoxycarbonyl, isopropoxycarbonylmethyl, isopropoxycarbonylethyl, butoxycarbonyl, butoxycarbonyl methyl, butoxycarbonylethyl, isobutoxycarbonyl, isobutoxycarbonylmethyl, isobutoxycarbonylethyl, pentyloxycarbonyl, pentyloxycarbonylmethyl, pentyloxycarbonylethyl, 2-methylbutoxycarbonyl, 2-methylbutoxycarbonylmethyl, 2-methylbutoxycarbonylethyl, 3-methylbutoxycarbonyl, 3-methylbutoxycarbonylmethyl, 3-methylbutoxycarbonylethyl, 2,2-dimethylpropoxycarbonyl, 2,2-dimethylpropoxycarbonylmethyl, 2,2-dimethylpropoxycarbonylethyl, hexyloxycarbonyl, hexyloxycarbonylmethyl, hexyloxycarbonylethyl, Further suitable examples of compounds include those where R⁶ is selected from the group consisting of —C₀₋₂alkyleneC(O)OR⁷, preferably where R⁶ is C(O)OR⁷, also include those where R⁷ is selected from the group consisting of C₀₋₆ alkylphenyl, for example phenyl which in turn is optionally substituted to form, for example 4-methoxy phenyl, 4-trifluoromethyl phenyl. Examples of such so substituted R⁶ groups include phenyloxycarbonyl, phenyloxycarbonylmethyl, and phenyloxy-carbonylethyl.

An even further group of suitable compounds include those where R⁶ is selected from the group consisting of —C₁₋₂alkyleneOC(O)NHR⁷, preferably where R⁶ is CH₂OC(O)NHR⁷, and where R⁷ is selected from the group consisting of C₁₋₈ alkyl; C₃₋₆ cycloalkyl for example cyclopropyl, cyclobutyl, cyclopentyl or cyclohexyl; C₁₋₆ haloalkyl for example trifluoromethyl, and trifluoroethyl; and C₀₋₆ alkylphenyl for example phenyl, phenylmethyl or phenylethyl which C₀₋₆ alkylphenyl is optionally substituted to form for example 4-methoxyphenyl, 4-trifluoromethylphenyl, 2,4-dichlorophenyl, 4-methoxyphenylmethyl, 4-trifluoromethylphenylmethyl, 2, 4-dichlorophenylmethyl, 4-methoxyphenylethyl, 4-trifluoromethylphenylethyl, or 2, 4-dichlorophenylethyl. Preferred are those compounds where R⁶ is selected from the group consisting of hydrogen, —C₀₋₂alkyleneR⁷ and —C₁₋₂alkyleneOC(O)R⁷ and where R⁷ is selected from the group consisting of C₁₋₈ alkyl.

Other suitable compounds are those when either one or more of R⁸ or R⁹ are phenyl, the phenyl group is optionally substituted with one or more substitutents selected from the group consisting of fluoro, chloro, methoxy or trifluoromethyl. Suitably when R⁸ and R⁹ together with the carbon to which they are attached may form a three- to six-membered carbocyclic, saturated ring it is preferred that the ring is a three membered ring.

Further suitable compounds include those where R⁷, R¹⁵ and R¹⁶ are, to where chemically possible, optionally substituted with one or more substituents selected from the group consisting of halo, C₁₋₄ alkyl, preferably methyl, C₃₋₆ cycloalkyl, preferably cyclopentyl, C₁₋₄ alkoxy, C₁₋₄ haloalkyl, preferably trifluoroethyl or trifluoromethyl, and S(O)ₙR¹⁰ for example methylsulphonyl or dimethyl amido sulphonyl. Examples of R⁷, R¹⁵ and R¹⁶ groups which have then been so substituted include for example branched alkyl groups such as 2-methylbutyl, 3-methylbutyl, substituted sulphonyl groups such as methylsulphonylmethyl, methylsulphonylethyl, dimethylamidosulphonylmethyl and dimethylamidosulphonylethyl and substituted phenyl groups such as 4-chlorophenyl, 4-nitrophenyl, 4-fluorophenyl, 4-methoxyphenyl, 2,4-dichlorophenyl, 4-chlorophenylmethyl, 4-nitrophenylmethyl, 4-fluorophenylmethyl, 4-methoxyphenyl methyl, 2,4-dichlorophenylmethyl, 4-chlorophenylethyl, 4-nitro phenyl ethyl, 4-fluorophenylethyl, 4-methoxyphenylethyl, and 2,4-dichlorophenylethyl.

Suitably when R¹⁵ and R¹⁶ together with the nitrogen to which they are attached form a three- to seven-membered saturated or unsaturated heterocyclic ring optionally containing one or more further N, O or S atoms it is preferred that the ring is a five- or six-membered ring, is saturated and comprises one further heteroatom selected from N, O or S. Suitable examples of such rings include pyrrolidinyl, pyrazolidinyl, imidazolinyl, thiazolidinyl, isoxazolidinyl, piperidinyl, piperazinyl, morpholinyl, or thiomorpholinyl. Preferred rings include pyrrolidinyl, thiazolidinyl, morpholinyl, or thiomorpholinyl. Such rings may optionally be further substituted with one or more groups, preferably selected from the group consisting of oxo, C(O)OH, halo for example fluoro or chloro, and C₁₋₄ alkyl for example methyl or ethyl preferably methyl. For example any heterocyclic sulphur atoms may be optionally substituted with one or more oxo groups to form for example 1,1-dioxothiazolidinyl or 1,1-dioxothiomorpholinyl substitutents.

Preferred substituted 1-N-arylpyrazole Formula (X) compounds

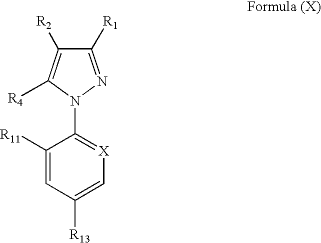

are those wherein

R₁ is cyano, methyl, or halo;

R₂ is S(O)ₙR₃, 4,5-dicyanoimidazol-2-yl, or haloalkyl;

R₃ is C₁₋₆alkyl or C₁₋₆haloalkyl;

R₄ is hydrogen, halo, NR₅R₆, S(O)ₘR₇, C(O)R₇, C(O)O—R₇, C₁₋₆alkyl, C₁₋₆haloalkyl —OR₈ or —N═C(R₉) (R₁₀);

R₅ and R₆ are each independently hydrogen, C₁₋₆alkyl, C₁₋₆haloalkyl, C(O)C₁₋₆alkyl, alkoxycarbonyl or S(O)CF₃, or R₅ and R₆ may together form a divalent alkylene radical which may be interrupted by one or two divalent hetero atoms such as oxygen or sulphur The ring formed by the divalent alkylene radical representing R₅ and R₆, as well as the nitrogen atom to which R₅ and Re are attached, may be generally a 5-, 6- or 7-membered ring.

R₇ is C₁₋₆alkyl or C₁₋₆haloalkyl;

R₈ is hydrogen, C₁₋₆alkyl or C₁₋₆haloalkyl;

P₉ is hydrogen or C₁₋₆alkyl;

R₁₀ is phenyl or heteroaryl optionally substituted with one or more halogen atoms or a member of the group consisting of OH, —O—C₁₋₆alkyl, S—C₁₋₆alkyl, cyano and C₁₋₆alkyl;

R₁₁ and R₁₂ are each independently hydrogen, halo, cyano, or NO₂;

R₁₃ is halo, C₁₋₆haloalkyl, C₁₋₆haloalkoxy, S(O)_{q}CF₃ or SF₅;

m, n, q and r represent, independently of each other, an integer equal to 0, 1 or 2;

X is a trivalent nitrogen atom or C—R₁₂, the other three valency positions of the carbon atom forming part of the aromatic ring,

with the proviso that when R₁ is methyl, either R₃ is C₁₋₆haloalkyl, R₄ is NH₂, R₁₁ is chloro, R₁₃ is CF₃ and X is N; or R₂ is 4,5-dicyanoimidazol-2-yl, R₄ is chloro, R₁₁ is chloro, R₁₃ is CF₃ and X is —C-chloro.

More preferred Formula (X) compounds are those where

R₁ is cyano or methyl;

R₂ is S(O)ₙR₃;

R₃ is C₁₋₆alkyl or C₁₋₆haloalkyl;

R₄ is hydrogen, halo, —NR₅R₆, —S(O)ₘR₇, C(O)R₇, C₁₋₆alkyl, C₁₋₆haloalkyl or —OR₆ or —N═C(R₉)(R₁₀);

R₅ and R₆ are each independently hydrogen, C₁₋₆alkyl, C₁₋₆haloalkyl, C(O) C₁₋₆alkyl or S(O) CF₃; or R₅ and R₆ may together form a divalent alkylene radical which may be interrupted by one or two divalent hetero atoms such as oxygen or sulphur;

R₇ is C₁₋₆alkyl or CO₁₋₆haloalkyl;

R₈ is hydrogen, C₁₋₆alkyl or C₁₋₆haloalkyl;

R₉ is hydrogen or C₁₋₆alkyl;

R₁₀ is phenyl or heteroaryl optionally substituted with one or more halo, OH, —O—C₁₋₆alkyl, S—C₁₋₆alkyl, cyano, or C₁₋₆alkyl;

R₁₁ and R₁₂ are each independently hydrogen or halo;

R₁₃ is halo, C₁₋₆haloalkyl, C₁₋₆haloalkoxy, S(O)_{q}CF₃, or SF₅;

m, n, q and r represent, independently of each other, an integer equal to 0, 1 or 2; and

X is a trivalent nitrogen atom or a radical C—R₁₂, the other three valency positions of the carbon atom forming par of the aromatic ring;

with the proviso that when R₁ is methyl, then R₃ is haloalkyl, R₄ is NH₂, R₁₁ is Cl, R₁₃ is CF₃ and X is N.

Formula (X) compounds in which R₁ is cyano will be selected most particularly. Compounds in which R₂ is S(O)ₙR³, preferably with n=1, R₃ preferably being CF₃ or C₁₋₆alkyl, for example methyl or ethyl, or alternatively n=0, R₃ preferably being CF₃, as well as those in which X═C—R₁₂, R₁₂ being a halogen atom, will also be selected. Compounds in which R₁₁ is a halogen atom and those in which R₁₃ is C₁₋₆haloalkyl, preferably CF₃, are also preferred. Within the context of the present invention, compounds which combine two or more of these characteristics will advantageously be selected.

A preferred class of Formula (X) compounds consists of compounds such that R₁ is cyano, R₃ is C₁₋₆haloalkyl, R₄ is NH₂, R₁₁ and R₁₂ are, independently of each other, a halogen atom, and R₁₃ is C₁₋₆haloalkyl, Preferably also, X is C—R₁₂.

In these compounds, R₃ preferably represents CF₃ or ethyl.

Most preferred, is a Formula (X) compound wherein R₁ is cyano; R₂ is —S(O)CF₃, R₄ is NH₂, R₁₁ and R₁₂ are both chloro, R₁₃ is CF₃, and X is CR₁₂, alternatively 5-amino-1-(2,6-dichloro-4-trifluoromethyl-phenyl)-4-trifluoromethanesulfinyl-1H-pyrazole-3-carbonitrile, the common name of which is fipronil.

A further group of preferred alpha substituted 2-benzyl imidazoles are Formula (1C) compounds

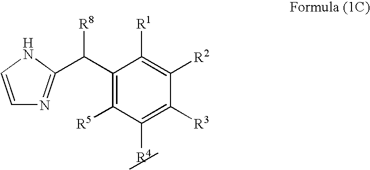

wherein R¹ to R⁵ are selected from hydrogen, halo, C₁₋₄ alkyl, C₁₋₄ haloalkyl and cyano, and R⁸ is C₁₋₃ alkyl Preferably, at least two of R¹ to R⁵ are hydrogen, and more preferably at least three of R¹ to R⁵ are hydrogen. Preferably, the groups from R¹ to R⁵ that are not hydrogen are selected from chloro, fluoro, methyl, ethyl, difluoromethyl and trifluoromethyl, and more preferably from fluoro, chloro and methyl. Preferably R⁸ is methyl or ethyl, and more preferably R⁸ is methyl.

A further group of preferred alpha substituted 2-benzyl imidazoles are Formula (1D) compounds

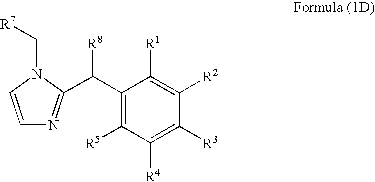

wherein R¹ to R⁵ are selected from hydrogen, halo, C₁₋₄ alkyl, C₁₋₄ haloalkyl and cyano, R⁷ is phenyl optionally substituted by one or more groups selected from cyano, nitro, halo, formyl, hydroxy, C(O)OH, C₁₋₄ alkyl, C₂₋₄ alkenyl, C₂₋₄ alkynyl, C₃₋₆ cycloalkyl, C₁₋₄ alkyleneC₃₋₆ cycloalkyl, C₁₋₄ alkoxy, —C(O)OC₁₋₄ alkyl, C₁₋₄ haloalkyl, C₁₋₄ haloalkoxy, pyrazolyl, triazolyl, amino, C₁₋₄ alkylamino, and C₁₋₄ dialkylamino, and R⁸ is C₁₋₃ alkyl. Preferably, at least two of R¹ to R⁵ are hydrogen, and more preferably at least three of R¹ to R⁵ are hydrogen. Preferably, the groups from R¹ to R⁵ that are not hydrogen are selected from chloro, fluoro, methyl, ethyl, difluoromethyl and trifluoromethyl, and more preferably from fluoro, chloro and methyl. Preferably R⁷ is phenyl optionally substituted by one or two groups selected from cyano, chloro, fluoro, hydroxy, C₁₋₃ alkyl, C₁₋₃ alkoxy and C₁₋₂ haloalkyl. Preferably R⁸ is methyl or ethyl, and more preferably R⁸ is methyl.

A further group of preferred alpha substituted 2-benzyl imidazoles are Formula (1E) compounds

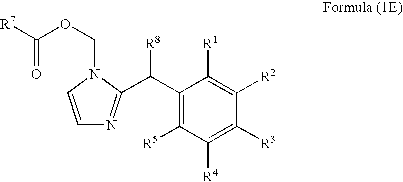

wherein R¹ to R⁵ are selected from hydrogen, halo, C₁₋₄ alkyl, C₁₋₄ haloalkyl and cyano, R⁷ is selected from C₁₋₃alkylenephenyl optionally substituted by on the phenyl ring by one or more groups selected from cyano, halo, hydroxy, C(O)OH, C₁₋₄ alkyl, C₃₋₆ cycloalkyl, C₁₋₄ alkyleneC₃₋₆ cycloalkyl, C₁₋₄ alkoxy, —C(O)OC₁₋₄ alkyl, C₁₋₄ haloalkyl, and C₁₋₄ haloalkoxy, C₁₋₈ alkyl optionally substituted by one or two C₁₋₄ alkoxy groups, C₃₋₆ cycloalkyl, C₁₋₃alkyleneC₃₋₆cycloalkyl₅, and C₁₋₆ haloalkyl, and R⁸ is C₁₋₃ alkyl. Preferably, at least two of R¹ to R⁵ are hydrogen, and more preferably at least three of R¹ to R⁵ are hydrogen. Preferably, the groups from R¹ to R⁵ that are not hydrogen are selected from chloro, fluoro, methyl, ethyl, difluoromethyl and trifluoromethyl, and more preferably from fluoro, chloro and methyl. Preferably R⁷ is C₁₋₈alkyl or C₁₋₆haloalkyl. Preferably R⁸ is methyl or ethyl, and more preferably R⁸ is methyl.

A further group of preferred alpha substituted 2-benzyl imidazoles are Formula (1F) compounds

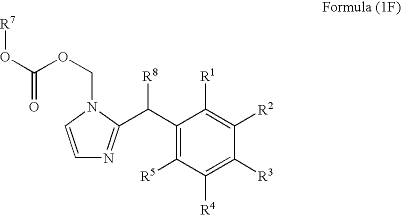

wherein R¹ to R⁵ are selected from hydrogen, halo, C₁₋₄ alkyl, C₁₋₄ haloalkyl and cyano, R⁷ is selected from C₁₋₃alkylenephenyl optionally substituted by on the phenyl ring by one or more groups selected from cyano, halo, hydroxy, C(O)OH, C₁₋₄ alkyl, C₃₋₆ cycloalkyl, C₁₋₄ alkyleneC₃₋₆ cycloalkyl, C₁₋₄ alkoxy, —C(O)OC₁₋₄ alkyl, C₁₋₄ haloalkyl, and C₁₋₄ haloalkoxy, C₁₋₈ alkyl optionally substituted by one or two C₁₋₄ alkoxy groups, C₃₋₆ cycloalkyl, C₁₋₃alkyleneC₃₋₆cycloalkyl, and C₁₋₆ haloalkyl, and R⁸ is C₁₋₃ alkyl. Preferably, at least two of R¹ to R⁵ are hydrogen, and more preferably at least three of R¹ to R⁵ are hydrogen. Preferably, the groups from R¹ to R⁵ that are not hydrogen are selected from chloro, fluoro, methyl, ethyl, difluoromethyl and trifluoromethyl, and more preferably from fluoro, chloro and methyl. Preferably R⁷ is C₁₋₈alkyl or C₁₋₆haloalkyl. Preferably R⁸ is methyl or ethyl, and more preferably R⁸ is methyl.

A further group of preferred alpha substituted 2-benzyl imidazoles are Formula (1G) compounds

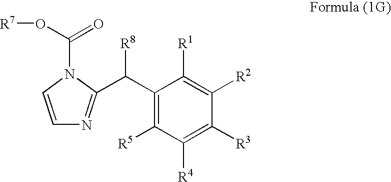

wherein R¹ to R⁵ are selected from hydrogen, halo, C₁₋₄ alkyl, C₁₋₄ haloalkyl and cyano, R⁷ is selected from C₁₋₃alkylenephenyl optionally substituted by on the phenyl ring by one or more groups selected from cyano, halo, hydroxy, C(O)OH, C₁₋₄ alkyl, C₃₋₆ cycloalkyl, C₁₋₄ alkyleneC₃₋₆ cycloalkyl, C₁₋₄ alkoxy, —C(O)OC₁₋₄ alkyl, C₁₋₄ haloalkyl, and C₁₋₄ haloalkoxy, C₁₋₈ alkyl optionally substituted by one or two C₁₋₄ alkoxy groups, C₃₋₆ cycloalkyl, C₁₋₃alkyleneC₃₋₆cycloalkyl, and C₁₋₆ haloalkyl, and R⁸ is C₁₋₃ alkyl. Preferably, at least two of R¹ to R⁵ are hydrogen, and more preferably at least three of R¹ to R⁵ are hydrogen Preferably, the groups from R¹ to R⁵ that are not hydrogen are selected from chloro, fluoro, methyl, ethyl, difluoromethyl and trifluoromethyl, and more preferably from fluoro, chloro and methyl. Preferably R⁷ is C₁₋₈alkyl or C₁₋₆haloalkyl, and more preferably R⁷ is isobutyl. Preferably R⁸ is methyl or ethyl, and more preferably R⁸ is methyl.

More preferred alpha benzyl substituted imidazoles employed in the combination of the invention are selected from the group consisting of
2-[(2,3-dimethylphenyl)(methoxy)methyl]-1H-imidazole;
2-[1-(2,5-dimethylphenyl)ethyl]-1H-imidazole;
2-[1-(2,4-dimethylphenyl)ethyl]-1H-imidazole;
2-[1-(3,4-dimethylphenyl)ethyl]-1H-imidazole;
2-{1-[2-(trifluoromethyl)phenyl]ethyl}-1H-imidazole;
(2,3-dimethylphenyl)(1H-imidazol-2-yl)methanol;
2-[1-(2,3-dimethylphenyl)ethyl]-1H-imidazole;
{2-[1-(2,3-dimethylphenyl)ethyl]-1H-imidazol-1-yl}methyl pivalate;
{2-[1-(2,3-dimethylphenyl)ethyl]-1H-imidazol-1-yl}methyl propionate;
{2-[1-(2,3-dimethylphenyl)ethyl]-1H-imidazol-1-yl}methyl 3-methylbutanoate;
{2-[1-(2,3-dimethylphenyl)ethyl]-1H-imidazol-1-yl}methyl butyrate;
{2-[1-(2,3-dimethylphenyl)ethyl]-1H-imidazol-1-yl}methyl 3-cyclopentylpropanoate;
{2-[1-(2,3-dimethylphenyl)ethyl]-1H-imidazol-1-yl}methyl heptanoate;
{2-[1-(2,3-dimethylphenyl)ethyl]-1H-imidazol-1-yl}methyl pentanoate;
2-[1-(4-chloro-3-methylphenyl)ethyl]-1H-imidazole;
2-[1-(3,5-dimethylphenyl)ethyl]-1H-imidazole;
1-benzyl-2-[1-(2,3-dimethylphenyl)ethyl]-1H-imidazole;
{2-[1-(2,3-dimethylphenyl)ethyl]-1H-imidazol-1-yl}methyl 4-methoxybenzyl carbonate;
1-(cyclopropylmethyl)-2-[1-(2,3-dimethylphenyl)ethyl]-1H-imidazole;
2-[1-(2,3-dimethylphenyl)ethyl]-1-methyl-1H-imidazole;
cyclopropylmethyl {2-[1-(2,3-dimethylphenyl)ethyl]-1H-imidazol-1-yl}methyl carbonate;
{2-[1-(2,3-dimethylphenyl)ethyl]-1H-imidazol-1-yl}methyl 3-methylbutyl carbonate;
{2-[1-(2,3-dimethylphenyl)ethyl]-1H-imidazol-1-yl}methyl isopropyl carbonate;
cyclobutyl {2-[1-(2,3-dimethylphenyl)ethyl]-1H-imidazol-1-yl}methyl carbonate;
{2-[1-(2,3-dimethylphenyl)ethyl]-1H-imidazol-1-yl}methyl 2,2,2-trifluoroethyl carbonate;
2-[1-(2,3-dimethylphenyl)ethyl]-1-ethyl-1H-imidazole;
2-[1-(2,3-dimethylphenyl)ethyl]-1-(4-methoxybenzyl)-1H-imidazole;
2-[1-(2,3-dimethylphenyl)ethyl]-1-(methoxymethyl)-1H-imidazole;
2-[1-(2,3-dimethylphenyl)ethyl]-1-[4-(trifluoromethyl)benzyl]-1H-imidazole;
4-fluorophenyl 2-[1-(2,3-dimethylphenyl)ethyl]-1H-imidazole-1-carboxylate;
isobutyl 2-[1-(2,3-dimethylphenyl)ethyl]-1H-imidazole-1-carboxylate;
isopropyl 2-[1-(2,3-dimethylphenyl)ethyl]-1H-imidazole-1-carboxylate; and
2-[1-(3-methylphenyl)ethyl]-1H-imidazole,
or a pharmaceutically or veterinarily acceptable salt or prodrug thereof.

More preferred alpha benzyl substituted imidazoles employed in the combination of the present invention are selected from the group consisting of
2-[1-(2,3-dimethylphenyl)ethyl]-1H-imidazole;
2-[(1S)-1-(2,3-dimethylphenyl)ethyl]-1H-imidazole;
2-[(1R)-1-(2,3-dimethylphenyl)ethyl]-1H-imidazole;
{2-[1-(2,3-dimethylphenyl)ethyl]-1H-imidazol-1-yl}methyl pivalate;
{2-[(1S)-1-(2,3-dimethylphenyl)ethyl]-1H-imidazol-1-yl}methylpivalate;
{2-[(1R)-1-(2,3-dimethylphenyl)ethyl]-1H-imidazol-1-yl}methylpivalate;
{2-[1-(2,3-dimethylphenyl)ethyl]-1H-imidazol-1-yl}methyl propionate;
{2-[1-(2,3-dimethylphenyl)ethyl]-1H-imidazol-1-yl}methyl 3-methylbutanoate;
{2-[1-(2,3-dimethylphenyl)ethyl]-1H-imidazol-1-yl}methyl butyrate;
{2-[1-(2,3-dimethylphenyl)ethyl]-1H-imidazol-1-yl}methyl 3-cyclopentylpropanoate;
{2-[1-(2,3-dimethylphenyl)ethyl]-1H-imidazol-1-yl}methyl heptanoate;
{2-[1-(2,3-dimethylphenyl)ethyl]-1H-imidazol-1-yl}methyl pentanoate;
2-{1-[2-(trifluoromethyl)phenyl]ethyl}-1H-imidazole;
2-[1-(2,5-dimethylphenyl)ethyl]-1H-imidazole;
2-[1-(4-chloro-3-methylphenyl)ethyl]-1H-imidazole;
2-[1-(3,5-dimethylphenyl)ethyl]-1H-imidazole;
1-benzyl-2[1-(2,3-dimethylphenyl)ethyl]-1H-imidazole;
{2-[1-(2,3-dimethylphenyl)ethyl]-1H-imidazol-1-yl}methyl 4-methoxybenzyl carbonate;
1-(cyclopropylmethyl)-2-[1-(2,3-dimethylphenyl)ethyl]-1H-imidazole;
2-[1-(2,3-dimethylphenyl)ethyl]-1-methyl-1H-imidazole;
cyclopropylmethyl {2-[1-(2,3-dimethylphenyl)ethyl]-1H-imidazol-1-yl}methyl carbonate;
{2-[1-(2,3-dimethylphenyl)ethyl]-1H-imidazol-1-yl}methyl 3-methylbutyl carbonate;
{2-[1-(2,3-dimethylphenyl)ethyl]-1H-imidazol-1-yl}methyl isopropyl carbonate;
cyclobutyl {2-[1-(2,3-dimethylphenyl)ethyl]-1H-imidazol-1-yl}methyl carbonate;
{2-[1-(2,3-dimethylphenyl)ethyl]-1H-imidazol-1-yl}methyl 2,2,2-trifluoroethyl carbonate;
2-[1-(2,3-dimethylphenyl)ethyl]-1-ethyl-1H-imidazole;
2-[1-(2,3-dimethylphenyl)ethyl]-1-(4-methoxybenzyl)-1H-imidazole;
2-[1-(2,3-dimethylphenyl)ethyl]-1-(methoxymethyl)-1H-imidazole;
2-[1-(2,3-dimethylphenyl)ethyl]-1-[4-(trifluoromethyl)benzyl]-1H-imidazole;
4-fluorophenyl 2-[1-(2,3-dimethylphenyl)ethyl]-1H-imidazole-1-carboxylate;
isobutyl 2-[l -(2,3-dimethylphenyl)ethyl]-1H-imidazole-1-carboxylate;
isopropyl 2-[1-(2,3-dimethylphenyl)ethyl]-1H-imidazole-1-carboxylate; and
2-[1-(3-methylphenyl)ethyl]-1H-imidazole,
or a pharmaceutically or veterinarilly acceptable salt or prodrug thereof.

Even more preferred alpha benzyl substituted imidazole compounds employed in the combination of the present invention are 2-[1-(2,3-dimethylphenyl)ethyl]-1H-imidazole, {2-[l -(2,3-dimethylphenyl)ethyl]-1H-imidazol-1-yl}methyl pivalate, 2-[(1S)-1-(2,3-dimethylphenyl)ethyl]-1H-imidazole, and {2-[(1S)-1-(2,3-dimethylphenyl)ethyl]-1H-imidazol-1-yl}methylpivalate, or a pharmaceutically or veterinarily acceptable salt or prodrug thereof.

The most preferred alpha benzyl substituted imidazoles employed in the combination of the present invention are 2-[(1S)-1-(2,3-dimethylphenyl)ethyl]-1H-imidazole and {2-[(1S)-1-(2,3-dimethylphenyl)ethyl]-1H-imidazol-1-yl}methylpivalate, or a pharmaceutically or veterinarily acceptable salt or prodrug thereof.

### BRIEF DESCRIPTION

Included within the scope of the present invention are all stereoisomers such as enantiomers and diasteromers, all geometric isomers of the compounds of Formula (1) and Formula (X) compounds exhibiting more than one type of isomerism, and mixtures of one or more thereof.

It is to be understood that Formula (1) compounds may contain one or more asymmetric carbon atoms, thus compounds of the invention can exist as two or more stereoisomers. In particular it will be understood that when R⁸ and R⁹ are different substitutents a stereocenter exists at the carbon atom to which they are attached—the benzylic carbon. Suitable compounds for use in this invention include those where the absolute stereochemistry at the benzylic carbon has the “S configuration”. Further suitable compounds for use in this invention include those where the absolute stereochemistry at the benzylic carbon has the “R configuration”. Such stereoisomers can be resolved and identified by one skilled in the art using known techniques.

The present invention includes the individual stereoisomers of the Formula (1) compounds together with mixtures thereof.

Conventional techniques for the preparation/isolation of individual enantiomers include chiral synthesis from a suitable optically pure precursor, stereoselective synthesis from a prochiral precursor or resolution of the racemate (or the racemate of a salt or derivative) using, for example, fractional crystallization or chiral high pressure liquid chromatography (HPLC). Reference is made herein to “Enantiomers, Racemates and Resolutions.” J. Jacques and A. Collet, published by Wiley, NY, 1981; and “Handbook of Chiral Chemicals” chapter 8, Eds D. Ager and M. Dekker, ISBN:0-8247-1058-4.

Alternatively, the racemate (or a racemic precursor) may be reacted with a suitable optically active compound, for example, an alcohol, or, in the case where the Formula (1) compound contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine, Concentration of the eluant affords the enriched mixture.

Stereoisomeric conglomerates may be separated by conventional techniques known to those skilled in the art—see, for example, “Stereochemistry of Organic Compounds” by E L Eliel (Wiley, New York, 1994).

Geometric isomers may be separated by conventional techniques well known to those skilled in the art, for example, chromatography and fractional crystallization, In the Formula (1) and (X) compounds, the term ‘halo’ means a group selected from fluoro, chloro, bromo or iodo.

Alkyl, alkylene, alkenyl, alkynyl and alkoxy groups, containing the requisite number of carbon atoms, can be unbranched or branched. Examples of alkyl include methyl, ethyl, n-propyl, i-propyl, n-butyl, i-butyl, s-butyl and t-butyl. Examples of alkoxy include methoxy, ethoxy, n-propoxy, i-propoxy, n-butoxy, i-butoxy, s-butoxy and t-butoxy. Examples of alkylene include —CH₂—, —CH(CH₃)— and —C₂H₄—. Examples of cycloalkyl include cyclopropyl, cyclobutyl, cyclopentyl, cyclohexyl and cycloheptyl.

For the avoidance of doubt, it will be understood that throughout the application all references to pharmaceutically acceptable compounds includes references to veterinarily acceptable compounds or agriculturally acceptable compounds. Furthermore it will be understood that throughout the application all references to pharmaceutical activity includes references to veterinary activity or agricultural activity.

Pharmaceutically or veterinarily acceptable salts of the Formula (1) and (X) compounds include the acid addition and base salts thereof. Suitable acid addition salts are formed from acids, which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochoride/chloride, hydrobromide/bromide, hydroiodida/iodide, isethionate, lactate, laurate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Suitable base salts are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

The pharmaceutically, veterinarily and agriculturally acceptable acid addition salts of certain of the Formula (1) and (X) compounds may also be prepared in a conventional manner. For example, a solution of a free base may be treated with the appropriate acid, either neat or in a suitable solvent, and the resulting salt isolated either by filtration or by evaporation under reduced pressure of the reaction solvent. For a review on suitable salts, see “Handbook of Pharmaceutical Salts: Properties, Selection, and Use” by Stahl and Wermuth (Wiley-VCH, Weinheim, Germany, 2002).

The compounds of the invention may exist in both unsolvated and solvated forms. The term ‘solvate’ is used herein to describe a molecular complex comprising the compound of the invention and one or more pharmaceutically or veterinarily acceptable solvent molecules, for example, ethanol. The term ‘hydrate’ is employed when said solvent is water. Pharmaceutically or veterinarily acceptable solvates in accordance with the invention include those wherein the solvent of crystallization may be isotopically substituted, e.g. D₂O, d₆-acetone, d₆-DMSO.

Hereinafter and throughout the application all references to Formula (1) and (X) compounds include references to salts, solvates and complexes thereof and to solvates and complexes of salts thereof.

As stated, the invention includes all polymorphs of the Formula (1) and (X) compounds as hereinbefore defined Included within the scope of the invention are complexes such as clathrates, drug-host inclusion complexes wherein, in contrast to the aforementioned solvates the drug and host are present in stoichiometric or non-stoichiometric amounts. Also included are complexes of the drug containing two or more organic and/or inorganic components which may be in stoichiometric or non-stoichiometric amounts. The resulting complexes may be ionized, partially ionized, or non-ionized. For a review of such complexes, see J Pharm Sci, 64 (8), 1269-1288 by Haleblian (August 1975).

The present invention includes all pharmaceutically or veterinarily acceptable isotopically-labelled Formula (1) and (X) compounds wherein one or more atoms are replaced by atoms having the same atomic number, but an atomic mass or mass number different from the atomic mass or mass number to usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Within the scope of the invention are so-called ‘prodrugs’ of the Formula (1) and (X) compounds. Thus certain derivatives of Formula (I) and (X) compounds which may have little or no pharmacological activity themselves can, when administered into or onto the body of an animal, be converted by the host or parasite into Formula (1) and/or (X) compounds having the desired activity, for example, by hydrolytic or enzymatic cleavage. Such derivatives are referred to as ‘prodrugs’. It will be appreciated that certain Formula (1) and (X) compounds may themselves act as pro-drugs of other Formula (i) and (X) compounds, respectively. Further information on the use of prodrugs may be found in ‘Pro-drugs as Novel Delivery Systems, Vol. 14, ACS Symposium Series (T Higuchi and W Stella) and: ‘Bioreversible Carriers in Drug Design’, Pergamon Press, 1987 (ed E B Roche, American Pharmaceutical Association),

Prodrugs in accordance with the invention can, for example, be produced by replacing appropriate functionalities present in the Formula (l) and (X) compounds with certain moieties known to those skilled in the art as ‘pro-moieties’ as described, for example, in “Design of Prodrugs” by H Bundgaard (Elsevier, 1985).

Some examples of prodrugs in accordance with the invention include:
(a) where the compound of formula (l) contains a carboxylic acid functionality (—COOH), an ester thereof, for example, replacement of the hydrogen with (C₁-C₈)alkyl;
(b) where the Formula (1) compound contains an alcohol functionality (—OH), an ether thereof, for example, replacement of the hydrogen with (C₁-C₆)alkanoyloxy-methyl; and
(c) where the Formula (l) compound contains a primary or secondary amino functionality (—NH₂ or —NHR where R is not H), an amide thereof, for example, replacement of one or both hydrogen's with (C₁-C₁₀)alkanoyl.

Prodrugs in accordance with the invention can, for example, be produced by reacting Formula (1) compounds wherein R⁶ is H with certain moieties known to those skilled in the art as ‘pro-drug moieties’ as described, for example, in “Design of Prodrugs” by H Bundgaard (Elsevier, 1985); “Design and application of prodrugs,” Textbook of Drug Design and Discovery, (3^{rd} Edition), 2002, 410-458, (Taylor and Francis Ltd., London); and references therein Examples of substituents include: alkyl amines, aryl amines, amides, ureas, carbamates, carbonates, imines, enamines, imides, sulfenamides, and sulfonamides. The hydrocarbon portion of these groups contain C₁₋₆ alkyl, phenyl, heteroaryl such as pyridyl, C₂₋₆ alkenyl, and C₃₋₈ cycloalkyl; wherein each of the above groups may include one or more optional substituents where chemically possible independently selected from: halo; hydroxy; C₁₋₆ alkyl, C₁₋₆ haloalkyl and C₁₋₆ alkoxy.

Further examples of replacement groups in accordance with the foregoing example and examples of other prodrug types may be found in the aforementioned references.

A prodrug that is administered to a test animal and metabolized by the host according to the invention can be readily identified by sampling a body fluid for a Formula (I) compound.

Finally, certain Formula (1) compounds may themselves act as prodrugs of other Formula (I) compounds.

In a further aspect, the present invention provides processes for the preparation of a Formula (I) and (X) compound, or a pharmaceutically, veterinarily or agriculturally acceptable salt thereof, or a pharmaceutically, veterinarily or agriculturally acceptable solvate (including hydrate) of either entity, as illustrated below. Specifically the Formula (X) compound can be prepared according to one of the processes described in U.S. Pat. No. 5,232,940, or European Patent EP-A-0 295 117, incorporated in their entireties by reference herein, The processes for the preparation of Formula (I) compounds are fully described in PCT WO 2007/083207 and incorporated herein by reference.

The skilled person will appreciate that the compounds of the invention could be made by methods other than those herein described as incorporated herein by reference, by adaptation of the methods herein described and/or adaptation of methods known in the art, for example the art described herein, or using standard textbooks such as “Comprehensive Organic Transformations—A Guide to Functional Group Transformations”, R C Larock, Wiley-VCH (1999 or later editions).

Among the compounds of insect growth regulators which are contemplated for the combinations of the present invention include those which are juvenile insect growth regulators, e.g., methoprene (1-methyethyl (E,E)-11-methoxy-3,7,11-trimethyl-2,4-dodecadienoate), such as s-methoprene, azadirachtin, diofenolan, fenoxycarb, pyriproxyfen, kinoprene, hydroprene, cyromazine, lufenuron and the like. A preferred insect growth regulator is s-methoprene.

This invention also relates to a pharmaceutical composition comprising a Formula (1) compound, or a pharmaceutically or veterinarily acceptable salt thereof, or a pharmaceutically or veterinarily acceptable solvate of either entity, a Formula (X) compound or a pharmaceutically or veterinarily acceptable salt thereof, or a pharmaceutically or veterinarily acceptable solvate of either entity and optionally an insect growth regulator, such as s-methoprene together with a pharmaceutically or veterinarily acceptable diluent or carrier, which may be adapted for oral, parenteral or topical administration (e.g. spot-on, multi spot-on, pour-on, stripe-on, roll-on or comb-on). The invention also relates to a pharmaceutical composition comprising a Formula (1) compound, or a pharmaceutically or veterinarily acceptable salt thereof, or a pharmaceutically acceptable solvate of either entity, a Formula (X) compound or a pharmaceutically or veterinarily acceptable salt thereof, or a pharmaceutically or veterinarily acceptable solvate of either entity and optionally an insect growth regulator such as s-methoprene together with a pharmaceutically or veterinarily acceptable diluent or carrier, which may be adapted for delivery via a device, such as a dual chamber device permitting the simultaneous or sequential delivery of each compound in the case of a combination comprising e.g. a Formula (1) compound and a Formula (X) compound.

Pharmaceutical compositions suitable for the delivery of combinations of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

Compounds of the invention intended for pharmaceutical use may be administered as crystalline or amorphous products. They may be obtained, for example, as solid plugs, powders, or films by methods such as precipitation. crystallization, freeze drying, or spray drying:, or evaporative drying. Microwave or radio frequency drying may be used for this purpose.

The methods by which the compounds may be administered include oral administration by capsule, bolus, tablet, powders, lozenges, chews, multi and nanoparticulates, gels, solid solution, films, sprays, or liquid formulation. Liquid forms include suspensions, solutions, syrups, drenches and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet. Oral drenches are commonly prepared by dissolving or suspending the active ingredient in a suitable medium. The liquid forms can also be applied topically in accordance with the present invention as spot-ons, multi-spot-ons, pour-ons, stripe-ons or roll-ons or comb-ons, for example,

As described herein compounds of the present invention may be administered alone or in combination with one or more other compounds of the invention or in combination with one or more other drugs (or as any combination thereof). The compounds may be administered alone or in a formulation appropriate to the specific use envisaged, the particular species of host mammal being treated and the parasite involved. Generally, they will be administered as a formulation in association with one or more pharmaceutically or veterinarily acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form. In one embodiment, the present invention contemplates any glycol ether, preferably DPGMME and ethanol in an amount of 0%-40% (w/v) and preferably 20% (w/v) of solution comprising a Formula (1) compound, a Formula (X) compound, and optionally s-methoprene.

Compositions useful for oral administration may be prepared by mixing the active ingredient with a suitable finely divided diluent and/or disintegrating agent and/or binder, and/or lubricant etc. Other possible ingredients include anti-oxidants, colorants, flavoring agents, preservatives and taste-masking agents For oral dosage forms, depending on dose, the drugs may make up from 1 wt % to 80 wt % of the dosage form, more typically from 5 wt % to 60 wt % of the dosage form. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form.

Binders are generally used to impart cohesive qualities to a tablet formulation Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Examples of diluents include lactose (monohydrate, spray-dried monohydrate, anhydrous and the like), mannitol, xylitol, dextrose, sucrose, sorbitol, microcrystalline cellulose, starch and dibasic calcium phosphate dihydrate.

Oral formulations may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet.

Lubricants include magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt % of the tablet.

Exemplary tablets contain up to about 80% drugs, from about 10 wt % to about 90 wt % binder, from about 0 wt % to about 85 wt % diluent, from about 2 wt % to about 10 % disintegrant, and from about 0.25 wt % to about 10 wt % lubricant.

The formulation of tablets is discussed in “Pharmaceutical Dosage Forms: Tablets, Vol, 1”, by H. Lieberman and L. Lachman, Marcel Dekker, N.Y., 1980 (ISBN 0-8247-6918-X).

The compounds may be administered topically to the skin or mucosa, that is dermally or transdermally. This is a preferred method of administration and as such it is desirable to develop active compounds, which are particularly suited to such formulations. Typical formulations for this purpose include pour-on, spot-on, multi-spot-on, stripe-on, comb-on, roll-on, dip, spray, mousse, shampoo, powder formulation, gels, hydrogels, lotions, solutions, creams, ointments, dusting powders, dressings, foams, films, skin patches, wafers, implants, sponges, fibers, bandages and micro emulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci. 88 (10),. 955-958 by Finnin and Morgan (October 1999). Pour-on or spot-on formulations may be prepared by dissolving the active ingredients in an acceptable liquid carrier vehicle such as butyl digol, liquid paraffin or a non-volatile ester, optionally with the addition of a volatile component such as propan-2-ol or a glycol ether Alternatively, pour-on, spot-on or spray formulations can be prepared by encapsulation, to leave a residue of active agent on the surface of the animal, this effect may ensure that the Formula (1) and Formula (X) compounds have increased persistence of action and are more durable, for example they may be more water fast. Alternatively, the topical combinations can be administered through a delivery device such as a dual chamber device which contains in a first chamber an amount of a Formula (1) compound optionally in combination with suitable exipients, adjuvants, disintegrants and the like suitable for e.g. spot-on delivery and in a second chamber, a Formula (X) compound, optionally in combination with suitable exipients, adjuvants, disintegrants and the like suitable for e.g. spot-on delivery. The contemplated dual-chambered device would be actuated by a user to administer a simultaneous, sequential or staged dosage of the combination of the invention to an animal such as a dog or cat for the prevention, treatment or control of ticks, fleas and mites.

Topical formulations of the combination contemplated herein can comprise from 1.0 mg/kg to 50 mg/kg of a Formula (1) compound, and preferably 10 mg/kg to 30 mg/kg of a Formula (1) compound, and most preferably 15, 16, 17, 18, 19, 20, 21, 22, 23, 24 or 25 mg/kg of a Formula (1) compound together with from 1.0 mg/kg to 20 mg/kg of a Formula (X) compound, and preferably 5 mg/kg to 10 mg/kg of a Formula (X) compound, and most preferably 6 mg/kg, 6.3 mg/kg, 6.5 mg/kg, 6.7 mg/kg, 6.9 mg/kg or 7 mg/kg of a Formula (X) compound. If s-methoprene is included in the combination, then the invention contemplates from 1.0 mg/kg to 10 mg/kg of s-methoprene and preferably 5 mg/kg to 8 mg/kg and most preferably 6 mg/kg s-methoprene. Such amounts are considered veterinarily acceptable in accordance with the present invention.

The invention contemplates monthly administration of the described combinations which can be increased to every 2 to 3 months depending upon the presence of s-methoprene. That is, if s-methoprene is included in the combination, then such combination can be administered every 2 to 3 months or more.

The compositions suitable for spot-on application according to the invention can be prepared by conventional mixing means. The volume of the applied composition can be from 1.0 mL/kg to 4 mL/kg and preferably 1.3 mL/kg to 3 mL/kg and most preferably 1.33 mL/kg to 1.7 mL/kg, inclusive.

Surprisingly, the combinations of the present application provide efficacy against ticks in 8 hours or less, with repellency rates extending from several weeks up to one month. Thus, the present invention provides a combination of a Formula (1) compound and a Formula (X) compound, and optionally s-methoprene in veterinarily acceptable amount can achieve effective treatment, prevention and control of ticks on animals in 8 hours or less. Thus, in accordance with the present invention, 8 hour efficacy can be achieved with a dual combination of a Formula (1) compound and a Formula (X) compound, without s-methoprene.

Agents may be added to the formulations of the present invention to improve the persistence of such formulations on the surface of the animal to which they are applied, for example to improve their persistence on the coat of the animal. It is particularly preferred to include such agents in a formulation which is to be applied as a pour-on or spot-on formulation. Examples of such agents include acrylic copolymers and in particular fluorinated acrylic copolymers. A particular suitable reagent is the trademark reagent “Foraperle” (Redline Products Inc, Texas, USA).

Certain topical formulations may include unpalatable additives to minimize accidental oral exposure.

Injectable formulations may be prepared in the form of a sterile solution, which may contain other substances, for example enough salts or glucose to make the solution isotonic with blood. Acceptable liquid carriers include vegetable oils such as sesame oil, glycerides such as triacetin, esters such as benzyl benzoate, isopropyl myristate and fatty acid derivatives of propylene glycol, as well as organic solvents such as pyrrolidin-2-one and glycerol formal. The formulations are prepared by dissolving or suspending the active ingredients in the liquid carrier such that the final formulation contains from 0.01 to 10% by weight of the active ingredient.

Alternatively, the combinations can be administered parenterally, or by injection directly into the blood stream, muscle or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including micro needle) injectors, needle-free injectors and infusion techniques. Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as powdered a dried form to be used in conjunction with a suitable vehicle such as sterile, pyrogen-free water. The preparation of parenteral formulations under sterile conditions, for example, by lyophilisation, may readily be accomplished using standard pharmaceutical techniques well known to those skilled in the art. The solubility of compounds of formula (I) used in the preparation of parenteral solutions may be increased by the use of appropriate formulation techniques, such as the incorporation of solubility-enhancing agents.

Such formulations are prepared in a conventional manner in accordance with standard medicinal or veterinary practice.

These formulations will vary with regard to the weight of active compound contained therein, depending on the species of host animal to be treated, the severity and type of infection and the body weight of the host. For parenteral, topical and oral administration, typical dose ranges of the active ingredient are 0.01 to 100 mg per kg of body weight of the animal Preferably the range is 0.1 to 10 mg per kg.

Formulations may be immediate and/or modified controlled release. Controlled release formulations include modified release formulations including delayed-, sustained-, pulsed-, controlled, targeted, or programmed release. Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). Alternatively, compounds of the invention may be formulated as a solid, semi-solid, or thixotropic liquid for administration as an implanted depot providing modified release of the active compound. Examples of such formulations include drug-coated stents and PGLA microspheres.

As an alternative the combinations may be administered to a non-human animal with the feedstuff and for this purpose a concentrated feed additive or premix may be prepared for mixing with the normal animal feed.

All the aforementioned aqueous dispersions or emulsions or spraying mixtures can be applied, for example, to crops by any suitable means, chiefly by spraying, at rates which are generally of the order of about 100 to about 1,200 L of spraying mixture per hectare, but may be higher or lower (e.g., low or ultra-low to volume) depending upon the need or application technique. The compounds or compositions according to the invention are conveniently applied to vegetation and in particular to roots or leaves having pests to be eliminated. Another method of application of the compounds or compositions according to the invention is by chemigation, that is to say, the addition of a formulation containing the active ingredient to irrigation water. This irrigation may be sprinkler irrigation for foliar pesticides or it can be ground irrigation or underground irrigation for soil or for systemic pesticides.

The concentrated suspensions, which can for example be applied by spraying, are prepared so as to produce a stable fluid product which does not settle (fine grinding) and usually contain from about 10 to about 75% by weight of active ingredient, from about 0.5 to about 30% of surface-active agents, from about 0.1 to about 10% of thixotropic agents, from about 0 to about 30% of suitable additives, such as anti-foaming agents, corrosion inhibitors, stabilizers, penetrating agents, adhesives and, as the carrier, water or an organic liquid in which the active ingredient is poorly soluble or insoluble. Some organic solids or inorganic salts may be dissolved in the carrier to help prevent settling or as antifreezes for water.

The wettable powers (or powder for spraying) are usually prepared so that they contain from about 10 to about 80% by weight of active ingredient, from about 20 to about 90% of a solid carriers from about 0 to about 5% of a wetting agent, from about 3 to about 10% of a dispersing agent and, when necessary, from about 0 to about 80% of one or more stabilizers and/or other additives, such as penetrating agents, adhesives, anti-caking agents, colorants, or the like. To obtain these wettable powders, the active ingredient(s) is (are) thoroughly mixed in a suitable blender with additional substances which may be impregnated on the porous filler and is (are) ground using a mill or other suitable grinder. This produces wettable powders, the wettability and the suspendability of which are advantageous. They may be suspended in water to give any desired concentration and this suspension can be employed very advantageously in particular for application to plant foliage.

The water dispersible granules (granules which are readily dispersible in water) have compositions which are substantially close to that of the wettable powders. They may be prepared by granulation of formulations described for the wettable powders, either by a wet route (contacting finely divided active ingredient with the inert filler and a little water, e.g. 1 to 20% by weight, or with an aqueous solution of a dispersing agent or binder, followed by drying and screening), or by a dry route (compacting followed by grinding and screening).

Depending on the method of application or the nature of the composition or use thereof, the rates and concentrations of the formulated compositions may vary according. Generally speaking, the compositions for application to control arthropod, plant nematode, helminth or protozoan pests usually contain from about 0.00001% to about 95%, more particularly from about 0.0005% to about 50% by weight of one or more Formula (1) compounds, Formula (X) compound, and optionally s-methoprene, or pesticidally acceptable salts thereof, or of total active ingredients (that is to say the Formula (1) compounds, Formula (X) compound, or a pesticidally acceptable salt thereof, together with: other substances toxic to arthropods or plant nematodes, anthelmintics, anticoccidials, synergists, trace elements or stabilizers). The actual compositions employed and their rate of application will be selected to achieve the desired effect(s) by the farmer, livestock producer, medical or veterinary practitioner, pest control operator or other person skilled in the art.

The combinations of the invention may be combined with soluble macromolecular entities, such as cyclodextrin and suitable derivatives thereof or polyethylene glyco-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins.

Combinations of the invention can also be mixed with one or more biologically active compounds or agents including insecticides, acaricides, anthelmintics, fungicides, nematocides, antiprotozoals, bactericides, growth regulators, entomopathogenic bacteria, viruses or fungi to form a multi-component pesticide giving an even broader spectrum of pharmaceutical, veterinary or agricultural utility. Thus, the present invention also pertains to a composition Is comprising a biologically effective amount of compounds of the invention and an effective amount of at least one additional biologically active compound or agent and can further comprise one or more of surfactant, a solid diluent or a liquid diluent. Specific further active compounds include those described in Patent Publication No. WO05/090313, at pages 39 to 44.

It be may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a Formula (1) compound and separately a Formula (X) compound in accordance with the invention, and means for separately retaining said compositions, such as a container, divided bottle, or divided foil packet. An example of such a kit is the familiar blister pack used for the packaging of tablets, capsules and the like. Another example of such a kit is a dual chambered device described above.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

The compounds of the invention, i.e. those of Formula (1) and Formula (X), possess parasiticidal activity in humans, animals, insects and plants. They are particularly useful in the treatment of ectoparasites.

This invention also relates to a combination of Formula (1) compound and a Formula (X) compound, or a pharmaceutically or veterinarily acceptable salt thereof, or a pharmaceutically or veterinarily acceptable solvate of either entity, or a pharmaceutical composition containing any of the foregoing, for use as a medicament.

A further aspect of this invention relates to the use of a Formula (1) and (X) compound, or a pharmaceutically or veterinarily acceptable salt thereof, or a pharmaceutically or veterinarily acceptable solvate of either entity, for the manufacture of a medicament for the treatment of a parasitic infestation.

In one embodiment this invention is useful for the manufacture of a medicament for the treatment of a parasitic infestation in humans.

In one embodiment this invention is useful for the manufacture of a medicament for the treatment of a parasitic infestation in animals.

In one embodiment this invention is useful for the manufacture of a medicament for the treatment of a parasitic infestation in insects.

In one embodiment this invention is useful for the manufacture of a medicament for the treatment of a parasitic infestation in plants.

An even further aspect of this invention relates to a method of treating a parasitic infestation in a mammal which comprises treating said mammal with an effective amount of a Formula (1) compound, Formula (X) compound or a pharmaceutically or veterinarily acceptable salt thereof, or a pharmaceutically acceptable solvate of either entity, and optionally s-methoprene or a pharmaceutical or veterinary composition containing any of the foregoing.

A yet further aspect of this invention relates to a method of preventing a parasitic infestation in a mammal which comprises treating said mammal with an effective amount of a Formula (1) compound, Formula (X) compound or a pharmaceutically or veterinarily acceptable salt thereof, or a pharmaceutically acceptable solvate of either entity, and optionally s-methoprene or a pharmaceutical composition containing any of the foregoing.

In a still further embodiment this invention also relates to a method of controlling disease transmission in a mammal which comprises treating said mammal with an effective amount of a Formula (1) compound, Formula (X) lo compound or a pharmaceutically or veterinarily acceptable salt thereof, or a pharmaceutically or veterinarily acceptable solvate of either entity, and optionally s-methoprene or a pharmaceutical composition containing any of the foregoing.

According to another aspect of the present invention, there is provided a method for the control of arthropod, plant nematode or helminth pests at a locus which comprises the treatment of the locus (e.g. by application or administration) with an effective amount of a Formula (1) compound, Formula (X) compound or a pesticidally acceptable salt thereof.

For the avoidance of doubt, references herein to “treatment” as used herein includes references to curative, palliative and prophylactic treatment; references to “control” (of parasites and/or pests etc.) include kill, repel, expel, incapacitate, deter, eliminate, alleviate, minimize, and eradicate.

The combinations of the invention have utility in the control of arthropod pests. They may, in particular, be used in the fields of veterinary medicine, livestock husbandry and the maintenance of public health: against arthropods which are parasitic internally or externally upon vertebrates, particularly warm-blooded vertebrates, including man and domestic animals such as dogs, cats, cattle, sheep, goats, equines, swine, poultry and fish for example Acarina: including ticks (e.g., Ixodes spp. (e.g., I. scapularis), Boophilus spp. (e.g., B. microplus), Amblyomma spp. (e.g., A. variegatum), Hyalomma spp, (e.g., H. marginatum), Rhipicephalus spp. (e.g., R. appendiculatus), Haemaphysalis spp. (e.g., H. punctata), Dermacentor spp. (e.g., D. variabilis), Ornithodorus spp, (e.g., O. moubata)); mites (erg. Damalinia spp. (edge, D. bovis) Dermanyssus spp, (D. galinae), Sarcoptes spp. (e.g., S. scabiei), Psoroptes spp. (e.g., P. cuniculi), Chorioptes spp. (e.g., C. equi), Demodex spp. (e.g., D. canis), Eutrombicula spp. (E. sarcina), Otodectes spp. (e.g., O. cynotis), Cheyletiella spp. (C. yasguri)), specific further arthropod pests include those described in Patent Publication No. WO 20051090313; Diptera (e.g., Aedes spp., Anopheles spp., Muscidae spp. (e.g., Stomoxys calcitrans and Haematobia irritans), Hypoderma spp., Gastrophilus spp., Simulium spp.); Hemiptera (e.g., Triatoma spp.); Phthiraptera (e.g., Damalinia spp., and Linognathus spp.); Siphonaptera (e.g., Ctenocephalides spp.); Dictyoptera (e.g., Periplaneta spp. and Blatella spp.) and Hymenoptera (e.g., Monomorium pharaonis). The combinations of the present invention also have utility in the field of control of plant pests, soil inhabiting pests and other environmental pests.

The present invention is particularly useful in the control of arthropod pests in mammals, in particular humans and animals. Preferably this invention is useful in the control of arthropod pests in animals which includes livestock such as cattle, sheep, goats, equines, swine and companion animals such as dogs and cats. Most preferably this invention is useful in the control of arthropod pests in dogs and cats.

The combinations of the invention are of particular value in the control of arthropods which are injurious to, or spread or act as vectors of diseases in, man and domestic animals, for example those hereinbefore mentioned, and more especially in the control of ticks, mites, lice, fleas, midges and biting, nuisance and myiasis flies. They are particularly useful in controlling arthropods which are present inside domestic host animals or which feed in or on the skin or suck the blood of the animal, for which purpose they may be administered orally, parenterally, percutaneously or topically.

The combinations of the invention are of value for the treatment and control of the various lifecycle stages of parasites including egg, nymph, larvae, juvenile and adult stages.

According to another aspect of the present invention, there is provided a method for the control of arthropod pests of insects which comprises treatment of the insect with an effective amount of a Formula (1) compound, Formula (X) compound or a pesticidally acceptable salt thereof and optionally s-methoprene. Combinations of the present invention may also be used for the treatment of infections caused by mites, and in particular varoaa mites. In particular combinations of the present invention may also be used for the treatment of varoaa mite infection in bees.

According to another aspect of the present invention, there is provided a method for the control of arthropod pests of plants which comprises treatment of the plant with an effective amount of a Formula (1) compound, Formula (X) compound or a pesticidally acceptable salt thereof and optionally s-methoprene. The combinations of the invention also have utility in the control of arthropod pests of plants. The active compound is generally applied to the locus at which the arthropod infestation is to be controlled at a rate of about 0.005 kg to about 25 kg of active compound per hectare (ha) of locus treated, preferably 0.02 to 2 kg/ha. Under ideal conditions, depending on the pest to be controlled, the lower rate may offer adequate protection. On the other hand, adverse weather conditions and other factors may require that the active ingredient be used in higher proportions. For foliar application, a rate of 0.01 to 1 kg/ha may be used. Preferably, the locus is the plant surface, or the soil around the plant to be treated.

According to another aspect of the present invention, there is provided a method for the protection of timber which comprises treatment of the timber with an effective amount of a Formula (1) compound, Formula (X) compound or a pesticidally acceptable salt thereof. Combinations of the present invention are also valuable in the protection of timber (standing, felled, converted, stored or structural) from attack by sawflies or beetles or termites. They have applications in the protection of stored products such as grains, fruits, nuts, spices and tobacco, whether whole, milled or compounded into products, from moth, beetle and mite attack. Also protected are stored animal products such as skins, hair, wool and feathers in natural or converted form (e.g. as carpets or textiles) from moth and beetle attack; also stored meat and fish from beetle, mite and fly attack. Solid or liquid compositions for application topically to timber, stored products or household goods usually contain from about 0,00005% to about 90%, more particularly from about 0.001% to about 10%, by weight of one or more Formula (1) compounds or pesticidally acceptable salts thereof.

The liquid compositions of this invention may, in addition to normal agricultural use applications be used for example to treat substrates or sites infested or liable to infestation by arthropods (or other pests controlled by compounds of this invention) including premises, outdoor or indoor storage or processing areas, containers or equipment or standing or running water.

The present invention also relates to a method of cleaning animals in good health comprising the application to the animal of a combination of formula (1), formula (X) or a veterinarily acceptable salt and optionally s-methoprene. The purpose of such cleaning is to reduce or eliminate the infestation of humans with parasites carried by the animal and to improve the environment in which humans inhabit.

### Biological Assay

The biological activity of the compounds was tested against ticks and fleas using one or more of the test methods described below.

In vitro Studies

Contact assays can be conducted to assess parasiticidal activity. Test compound(s) can be dissolved in a solvent, e.g., isopropyl alcohol. Aliquots of the solution can then be added to glass vials with a known inner surface area (e.g., 34.5 cm²). The vials can be tilted and rolled while the solvent is evaporated thereby equally coating the vials with known concentrations of the test compound(s), e.g., 0.1 μg/cm², 1 μg/cm², or 10 μg/cm². Ticks, mites or fleas can then be added to the vials. Dead and non-moving ticks, mites or fleas can be counted at specified timed intervals (e.g., 4, 12 and 24 hours) to assess compound efficacy.

Alternatively, membrane blood feeding assays can be conducted to assess compound efficacy. Test compound(s) can be dissolved in a solvent, e.g., dimethylsulphoxide. An aliquot of the solution can be added to citrated bovine blood to achieve certain compound concentrations (e.g., 1 μg/mL, 5 μg/mL, or 10 μg/mL). A volume (e.g., 5 mL) of the blood can be pre-warmed to 37° C. and added to small petri-dish lids. The lids can be covered with a thin film to form a tight feeding membrane. Ticks, mites or fleas can be added to untreated glass vials which can be affixed to the petri-dish feeding membranes. Ticks, mites, or fleas are allowed to feed for a period of time (e.g., 2-hours). Dead and non-moving ticks, mites or fleas can be counted at specified durations (e.g., 2, 4, and 24 hours post feeding) to determine efficacy, e.g., [(number of dead and non-moving fleas)/total fleas)×100] of the test compound.

### In-vivo Studies

In-vivo studies were conducted in compliance with International Cooperation on Harmonisation of Technical Requirements for Registration of Veterinary Products (VICH) and Good Clinical Practice (GCP) principles and practices.

In one in-vivo study, topical spot-on formulations were prepared using dipropylene glycol monomethyl ether (DPGMME) and ethanol (80:20% v/v) and butylated hydroxyl anisol (BHA) 0.1% w/v. Control was vehicle. Treatment T02 was 30 mg/kg (150 mg/mL) compound (1A1); T03 was 20 mg/kg (100 mg/mL) compound (1A1), 6.7 mg/kg (33.5 mg/mL) fipronil, and 6 mg/kg (30 mg/mL) s-methoprene; T04 was 30 mg/kg (150 mg/mL) compound (1A1), 6.7 mg/kg (50.4 mg/mL) fipronil, and 6 mg/kg (45 mg/mL) s-methoprene; and T05 was 30 mg/kg (150 mg/mL) compound (1A1), 6.7 mg/kg (33.5 mg/mL) fipronil and 6 mg/kg (30 mg/mL) s-methoprene. A total of 40 beagle dogs (8 dogs/treatment group) were used. Dogs were individually housed. Treatments were administered on Day 0. On Day 30, dogs were infested with 100 Ctenocephalides felis unfed adult fleas. Dead and non-moving fleas were counted at 24 and 48 hours after infestation (Table 1). As shown in Table 1, compound (1A1) alone, lacks intrinsic flea activity.

TABLE 1
Geometric mean percent (%) efficacy against C. felis

| Day 30 | 24 h | 48 h |
|---|---|---|
| T02 | 12.7* | 10.3* |
| T03 | 100 | 100 |
| T04 | 100 | 100 |
| T05 | 99.2 | 100 |

*Values were not statistically significant from untreated controls (p = 0.63 at 24 hours and P = 0.36 at 48 hours)

In a separate in-vivo study, a topical spot-on formulation (T02) containing compound (1A1), fipronil, and s-methoprene was prepared in DPGMME and ethanol (80:20% v/v) and BHA (0.1% w/v). Final dosing concentrations were 20 mg/kg for compound (1A1), 6.7 mg/kg for fipronil, and 6 mg/kg for s-methoprene. This formulation was compared with a commercial product (Frontline Plus (T03)) which provides a 6.7 mg/kg fipronil and 6 mg/kg s-methoprene dose. A total of 30 mixed breed dogs (6 dogs/treatment group) were housed individually. Each dog was artificially infested with 100 C. felis unfed adult fleas on Days—2, 7, 14, 21, 28, 30, and 35. Treatments were administered on Day 0. Dead and non-moving flea counts were conducted at 24 and 48 hours after each infestation (Table 2).

TABLE 2
Geometric mean percent (%) efficacy against C. felis

| | Day 0 | Day 7 | Day 14 | Day 21 | Day 28 | Day 35 |
|---|---|---|---|---|---|---|
| 24 hours | | | | | | |
| T02 | 100.0 | 100.0 | 100.0 | 100 | 99.7 | 100 |
| T03 | 95.4 | 100.0 | 100.0 | 100 | 95.8 | 69.7 |
| 48 hours | | | | | | |
| T02 | 100.0 | 100.0 | 99.8 | 100.0 | 100.0 | 100 |
| T03 | 100.0 | 100.0 | 100.0 | 100.0 | 99.0 | 96.5 |

Unexpectedly, compound (1A1) in combination with fipronil and s-methoprene provided 30% greater efficacy within 24 hours of flea infestation at Day 35 than did the commercial product containing the same dose of fipronil and s-methoprene.

In another in-vivo study, a topical spot-on formulation (T02) containing compound (1A1) and fipronil was prepared in a solution of lauric acid (165 mg/mL), BHA (2 mg/mL), and N-methylpyrrolidone (NMP) (qs v/v). Final dosing concentrations were 20 mg/kg (150 mg/mL) compound (1A1) and 6,7 mg/kg (50 mg/mL) fipronil. This formulation was compared with a commercial product (Frontline Top Spot (T03)) which provides a 6.7 mg/kg fipronil dose. A total of 52 mixed breed and beagle dogs (8 dogs/treatment group) were housed individually. Dogs were each artificially infested with 50 adult Ixodes ricinus unfed adult ticks on days—2, 7, 14, 21, and 28. Treatments were administered on Day 0. Tick counts were obtained at 24 hours (repellency effect) and at 48 hours (acaricidal effect) following treatment on days 0, 7, 14, 21, and 28 (Table 3).

TABLE 3
Geometric mean percent (%) efficacy against I. ricinus

| Treatment | Day 0 | Day 7 | Day 14 | Day 21 | Day 28 |
|---|---|---|---|---|---|
| | 24 hour - repellent | | | | |
| T02 | 50.4 | 93.5 | 98.8 | 95.1 | 92.3 |
| T03 | 35.1 | 75.7 | 80.1 | 59.0 | 46.9 |
| | 48 hour - acaricidal | | | | |
| T02 | 86.1 | 97.8 | 99.2 | 99.2 | 99.1 |
| T03 | 57.5 | 96.5 | 97.7 | 93.5 | 84.1 |

The combination of compound (1A1) and fipronil was significantly better than fipronil alone in repelling ticks at 24 hours following infestation. At 48 hours, the combination had a quicker onset of efficacy and a longer duration of activity as an acaricide than fipronil alone.

### Octopamine Activity

Application of octopamine agonists to acarids (e.g., ticks and mites) causes distinct behavioural changes compared to untreated ticks Treated ticks become agitated and move constantly, this prevents ticks from attaching and feeding on a host animal to which the compound has been applied. Normal behaviour of ticks is to go into stasis when all other external stimuli are removed. Agitation and movement can be measured in vitro in the laboratory to predict efficacy and potency in vivo.

One skilled in the art could also determine agonist activity against insect octopamine receptors expressed in CHO cells by adapting the methods described in B. Maqueira, H. Chatwin, P. D. Evans, J. Neurochemistry, 2005, 94, 2, 547. Compound activity can be measured as an increase in cAMP by various methods known to a skilled person and can be recorded as %Vmax (Vmax maximal octopamine response) and EC₅₀.

We claim.

1. A composition comprising an effective amount of a) a compound of formula (1), or a pharmaceutically or veterinarily acceptable salt or prodrug thereof, 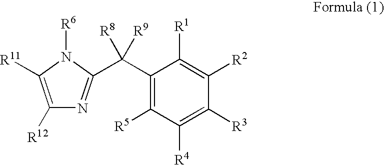
wherein
R¹, R², R³, R⁴, R⁵ are each independently hydrogen, halo, C₁₋₄ alkyl, C₁₋₄ alkoxy, or C₁₋₄ haloalkyl;
R⁶ is hydrogen, —C₀₋₂alkyleneR⁷, —C₁₋₂alkyleneOR⁷, —C₁₋₂alkyleneOC(O)R⁷, —C₁₋₂alkyleneOC(O)OR⁷, or —C₀₋₂alkyleneC(O)OR⁷;
R⁷ is hydrogen, C₁₋₆ alkyl, or C₁₋₄ alkylene(C₃₋₆cycloalkyl);
R⁸ and R⁹ are each independently hydrogen or C₁₋₄ alkyl, with the proviso that R⁸ and R⁹ are not both hydrogen; and
R¹¹ and R¹² are each independently hydrogen, C₁₋₂ alkyl, or C₁₋₂ alkoxy;
and b) an effective amount of a compound of Formula (X), or a pharmaceutically or veterinarily acceptable salt or prodrug thereof, 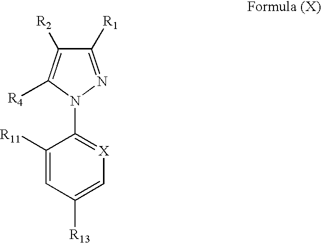
wherein
R₁ is cyano or methyl;
R₂ is S(O)ₙR₃;
R₃ is C₁₋₆alkyl or C₁₋₆haloalkyl;
R₄ is NH₂;
R₅ and R₆ are each independently hydrogen, C₁₋₆alkyl, C₁₋₆haloalkyl, C(O)C₁₋₆alkyl, or —S(O)ᵣCF₃;
R₇ is C₁₋₆-alkyl or C₁₋₆haloalkyl;
R₈ is hydrogen, C₁₋₆alkyl or C₁₋₆haloalkyl;
R₉ is hydrogen or C₁₋₆alkyl;
R₁₁ and R₁₂ are each independently hydrogen or halo;
R₁₃ is halo, C₁₋₆haloalkyl, C₁₋₆haloalkoxy, —S(O)_{q}CF₃, or SF₅;
n, q and r are integers each independently 0, 1 or 2; and
X is N or C—R₁₂;
with the proviso that when R₁ is methyl, then R₃ is C₁₋₆-haloalkyl, R₁₁ is chloro, R₁₃ is CF₃ and X is N;
and optionally c) an insect growth regulator which mimics juvenile hormones, and a pharmaceutical or veterinarily acceptable diluent or carrier.

2. The composition of claim 1, wherein said insect growth regulator is selected from the group consisting of s-methoprene, hydroprene, or pyriproxyfen.

3. A method for the prevention, treatment, or control of ticks, fleas, and mites in mammals comprising administering to said mammal an effective amount of a composition of claim 1.

4. The method of claim 3 wherein the composition is administered as a spot-on, multi-spot on, pour-on, stripe-on, or comb-on composition.

5. The method of claim 4 wherein the composition is admininstered as a spot-on composition and wherein the mammal is a dog or cat.

6. A composition comprising an effective amount of a) compound (1A1) 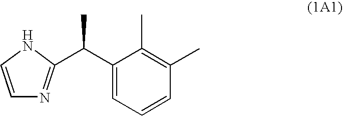
or a pharmaceutically or veterinarily acceptable salt or prodrug thereof, and b) an effective amount of a compound of Formula (X) 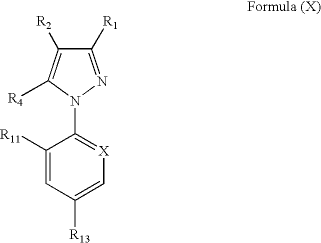
or a pharmaceutically or veterinarily acceptable salt or prodrug thereof, wherein
X is C—R₁₂;
R₁ is cyano;
R₂ is —S(O)CF₃;
R₄ is NH₂;
R₁₁ and R₁₂ are chloro; and
R₁₃ is CF₃;
and optionally c) an insect growth regulator which mimics juvenile hormones which is hydroprene, s-methoprene or pyriproxyfen; and a pharmaceutical or veterinarily acceptable diluent or carrier.

7. The composition of claim 6 wherein the insect growth regulator is s-methoprene.

8. The composition of claim 6 wherein said composition comprises from 1.0 mg/kg to 50 mg/kg of compound (1A1) and from 1.0 mg/kg to 20 mg/kg of the Formula (X) compound, and optionally 1 mg/kg to 10 mg/kg of s-methoprene.

9. A method for the prevention, treatment, or control of ticks, fleas, and mites in mammals comprising administering to said mammal an effective amount of a composition of claim 6.

10. The method of claim 9 wherein the composition is admininstered as a spot-on, multi-spot on, pour-on, stripe-on, or comb-on composition.

11. The method of claim 10 wherein the composition is admininstered as a spot-on composition.

12. The method of claim 9 wherein the mammal is a dog or cat.

13. A topical composition comprising an effective amount of a) a compound selected from the group consisting of
2-[(2,3-dimethylphenyl)(methoxy)methyl]-1H-imidazole;
2-[1-(2,5-dimethylphenyl)ethyl]-1H-imidazole;
2-[1-(2,4-dimethylphenyl)ethyl]-1H-imidazole;
2-[1-(3,4-dimethylphenyl)ethyl]-1H-imidazole;
{2-(1-[2-(trifluoromethyl)phenyl]ethyl}-1H-imidazole;
(2,3-dimethylphenyl)(1H-imidazol-2-yl)methanol;
2-[1-(2,3-dimethylphenyl)ethyl]-1H-imidazole;
{2-[1-(2,3-dimethylphenyl)ethyl]-1H-imidazol-1-yl}methyl pivalate;
{2-[1-(2,3-dimethylphenyl)ethyl]-1H-imidazol-1-yl}methyl propionate;
{2-[1-(2,3-dimethylphenyl)ethyl]-1H-imidazol-1-yl}methyl 3-methylbutanoate;
{2-[1-(2,3-dimethylphenyl)ethyl]-1H-imidazol-1-yl}methyl butyrate;
{2-[1-(2,3-dimethylphenyl)ethyl]-1H-imidazol-1-yl}methyl 3-cyclopentylpropanoate;
{2-[1-(2,3-dimethylphenyl)ethyl]-1H-imidazol-1-yl}methyl heptanoate;
{2-[1-(2,3-dimethylphenyl)ethyl]-1H-imidazol-1-yl}methyl pentanoate;
2-[1-(4-chloro-3-methylphenyl)ethyl]-1H-imidazole;
2-[1-(3,5-dimethylphenyl)ethyl]-1H-imidazole;
1-benzyl-2-[1-(2,3-dimethylphenyl)ethyl]-1H-imidazole;
{2-[1-(2,3-dimethylphenyl)ethyl]-1H-imidazol-1-yl}methyl 4-methoxybenzyl carbonate;
1-(cyclopropylmethyl)-2-[1-(2,3-dimethylphenyl)ethyl]-1H-imidazole;
2-[1-(2,3-dimethylphenyl)ethyl]-1-methyl-1H-imidazole;
cyclopropylmethyl {2-[1-(2,3-dimethylphenyl)ethyl]-1H-imidazol-1-yl}methyl carbonate;
{2-[1-(2,3-dimethylphenyl)ethyl]-1H-imidazol-1-yl}methyl 3-methylbutyl carbonate;
{2-[1-(2,3-dimethylphenyl)ethyl]-1H-imidazol-1-yl}methyl isopropyl carbonate;
cyclobutyl {2-[1-(2,3-dimethylphenyl)ethyl]-1H-imidazol-1-yl}methyl carbonate;
{2-[1-(2,3-dimethylphenyl)ethyl]-1H-imidazol-1-yl}methyl 2,2,2trifluoroethyl carbonate;
2-[1-(2,3-dimethylphenyl)ethyl]-1-ethyl-1H-imidazole;
2-[1-(2,3-dimethylphenyl)ethyl]-1-(4-methoxybenzyl)-1H-imidazole;
2-[1-(2,3-dimethylphenyl)ethyl]-1-(methoxymethyl)-1H-imidazole;
2-[1-(2,3-dimethylphenyl)ethyl]-1-[4-(trifluoromethyl)benzyl]-1H-imidazole;
4-fluorophenyl 2-[1-(2,3-dimethylphenyl)ethyl]-1H-imidazole-1-carboxylate;
isobutyl 2-[1-(2,3-dimethylphenyl)ethyl]-1H-imidazole-1-carboxylate;
isopropyl 2-[1-(2,3-dimethylphenyl)ethyl]-1H-imidazole-1-carboxylate;
2-[1-(3-methylphenyl)ethyl]-1H-imidazole,
2-[1-(2,3-dimethylphenyl)ethyl]-1H-imidazole;
2-[(1R-1-(2,3-dimethylphenyl)ethyl]-1H-imidazole;
{2-[1-(2,3-dimethylphenyl)ethyl]-1H-imidazol-1-yl}methyl pivalate;
{2-[(1S)-1-(2,3-dimethylphenyl)ethyl]-1H-imidazol-1-yl}methylpivalate;
{2-[(1R)-1-(2,3-dimethylphenyl)ethyl]-1H-imidazol-1-yl}methylpivalate;
{2-[1-(2,3-dimethylphenyl)ethyl]-1H-imidazol-1-yl}methyl propionate;
{2-[1-(2,3-dimethylphenyl)ethyl]-1H-imidazol-1-yl}methyl 3-methylbutanoate;
{2-[1-(2,3-dimethylphenyl)ethyl]-1H-imidazol-1-yl}methyl butyrate;
{2-[1-(2,3-dimethylphenyl)ethyl]-1H-imidazol-1-yl}methyl 3-cyclopentylpropanoate;
{2-[1-(2,3-dimethylphenyl)ethyl]-1H-imidazol-1-yl}methyl heptanoate;
{2-[1-(2,3-dimethylphenyl)ethyl]-1H-imidazol-1-yl}methyl pentanoate;
2-{1-[2-(trifluoromethyl)phenyl]ethyl}-1H-imidazole;
2-[1-(2,5-dimethylphenyl)ethyl]-1H-imidazole;
2-[1-(4-chloro-3-methylphenyl)ethyl]-1H-imidazole;
2-[1-(3,5-dimethylphenyl)ethyl]-1H-imidazole;
1-benzyl-2-[1-(2,3-dimethylphenyl)ethyl]-1H-imidazole;
{2-[1-(2,3-dimethylphenyl)ethyl]-1H-imidazol-1-yl}methyl 4-methoxybenzyl carbonate;
1-(cyclopropylmethyl)-2-[1-(2,3-dimethylphenyl)ethyl]-1H-imidazole;
2-[1-(2,3-dimethylphenyl)ethyl]-1-methyl-1H-imidazole;
cyclopropylmethyl {2-[1-(2,3-dimethylphenyl)ethyl]-1H-imidazol-1-yl}methyl carbonate;
{2-[1-(2,3-dimethylphenyl)ethyl]-1H-imidazol-1-yl}methyl 3-methylbutyl carbonate;
{2-[1-(2,3-dimethylphenyl)ethyl]-1H-imidazol-1-yl}methyl isopropyl carbonate;
cyclobutyl {2-[1-(2,3-dimethylphenyl)ethyl]-1H-imidazol-1-yl}methyl carbonate;
{2-[1-(2,3-dimethylphenyl)ethyl]-1H-imidazol-1-yl}methyl 2,2,2-trifluoroethyl carbonate;
2-[1-(2,3-dimethylphenyl)ethyl]-1-ethyl-1H-imidazole;
2-[1-(2,3-dimethylphenyl)ethyl]-1-(4-methoxybenzyl)-1H-imidazole;
2-[1-(2,3-dimethylphenyl)ethyl]-1-(methoxymethyl)-1H-imidazole;
2-[1-(2,3-dimethylphenyl)ethyl]-1-[4-(trifluoromethyl)benzyl]-1H-imidazole;
4-fluorophenyl 2-[1-(2,3-dimethylphenyl)ethyl]-1H-imidazole-1-carboxylate;
isobutyl 2-[1-(2,3-dimethylphenyl)ethyl]-1H-imidazole-1-carboxylate;
isopropyl 2-[1-(2,3-dimethylphenyl)ethyl]-1H-imidazole-1-carboxylate; and
2-[1-(3-methylphenyl)ethyl]-1H-imidazole,
or a pharmaceutically or veterinarily acceptable salt or prodrug thereof; and
b) 5-amino-1-(2,6-dichloro-4-trifluoromethyl-phenyl)-4-trifluoromethanesulfinyl-1H-pyrazole-3-carbonitrile, or a pharmaceutically or veterinarily acceptable salt or prodrug thereof;
and optionally c) an insect growth regulator which mimics juvenile hormones which is hydroprene, s-methoprene or pyriproxyfen;
and a pharmaceutical or veterinarily acceptable diluent or carrier.

14. A method for the prevention, treatment, or control of ticks, fleas, and mites in mammals comprising administering to said mammal an effective amount of a composition of claim 13 wherein said mammals are livestock and companion animals.

15. The method of claim 14 wherein the composition is admininstered as a spot-on, multi-spot on, pour-on, stripe-on, or comb-on composition.
